SEQUENCE LISTING (34 sequences)

SEQ ID NO: 1 - protein, 645 aa, Staphylococcus aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 2 - protein, 653 aa, Artificial Sequence
  0657n with a carboxy His-Tag
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKNLEHHHHHH

SEQ ID NO: 3 - protein, 569 aa, Artificial Sequence
  Truncated version of ORF0657n
MAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKS

SEQ ID NO: 4 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKEVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 5 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKKKDGTQQFYHYASSVEPARVIFTDSKPEIELGLQSGQFWRKFEVYEGDKKLPIKLVSYDTVKDYAYIRFSVSNGTKEVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 6 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKKKDGTQQFYHYASSVEPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELEKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 7 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKKKDGTQQFYHYASSVEPARVIFTDSKPEIELGLQSGQFWRKFEVYEGDKKLPIKLVSYDTVKDYAYIRFSVSNGTKEVKIVSSTHFNNKEEKYDYTLMVFAQPIYNSADKFKTEEDYKAEKLLAPYKKAKTLERQVYELNKIQDKLPEKLKAEYKKKLEDTKKALAEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 8 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKKKDGTQQFYHYASSVEPARVIFTDSKPEIELGLQSGQFWRKFEVYEGDKKLPIKLVSYDTVKDYAYIRFSVSNGTKEVKIVSSTHFNNKEEKYDYTLMEFAQPIYNSADKFKTEEDYKAEKLLAPYKKAKTLERQVYELEKIQDKLPEKLKAEYKKKLEDTKKALAEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 9 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKKKDGTQQFYHYASSVEPARVIFTKSKPEIELGLQSGQFWRKFEVYEGDKKLPIKLVSYDTDKDYAYIRFSVSNGTKEVKIVSSTHFNNKEEKYDYTLMEFAQPIYNSADKFKTEEDYKAEKLLAPYKKAKTLERQVYELEKIQDKLPEKLKAEYKKKLEDTKKALAEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 10 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKKKDGTQQFYHYASSVEPARVIFTKSKPEIELGLQSGSTWRKFEVYEGDKKLPIKLVSYDTDKDYAYIRFSVSNGTKEVKIVSSTHFNNKEEKYDYTLMVFAQPIYNSADKFKTEEDYKAEKLLAPYKKAKTLERQVYELEKIQDKLPEKLKAEYKKKLEDTKKALAEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 11 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKNDKGTQQFYHYASSVEPARVIFTKSKPIIELGLQSGQFWRKFEVYEGDKKLPIKLVSYDTDKDYAYIRFSVSNGTKEVKIVSSTHFNNKEEKYDYTLMVFAQPIYNSADKFKTEEDYKAEKLLAPYKKAKTLERQVYELEKIQDKLPEKLKAEYKKKLEDTKKALAEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 12 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKNDKGTQQFYHYASSVEPARVIFTKSKPIIELGLQSGQFWRKFEVYEGDKKLPIKLVSYDTDKDYAYIRFSVSNGTKEVKIVSSTHFNNKEEKYDYTLMVFAQPIYNSADKFKTEEDYKAEKLLAPYKKAKTLERQVYELEKIQDKLPEKLKAEYKKKLEQTKKALAEQVKSAITEFQNVQPTNEKMTDLQDAHYVVYESVENSESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 13 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKNDKGTQQFYHYASSVEPARVIFTKSKPIIELGLQSGQFWRKFEVYEGDKKLPIKLVSYDTDKDYAYIRFSVSNGTKEVKIVSSTHFNNKEEKYDYTLMVFAQPIYNSADKFKTEEDYKAEKLLAPYKKAKTLERQVYELEKIQDKLPEKLKAEYKKKLEQTKKALAEQVKSAITEFQNVQPTNEKMTDLQDAHYVVYESVENSESMMDTFVKHPIKTGMLNGKKYMVMETTNDDYWKDFMVEGKRVRTISKDAKNNTRTIIFPYVEGKALYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 14 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKEHSAPNSRPIDFEMKKKDGTQQFYHYASSVKPARVIFTDSKPEIELGLQSGQFWRKFEVYEGDKKLPVKLVSYDTVKDYAYIRFSVSNGTKAVKIVSSTHFNNKEEKYDYTLMEFAQPIYNSADKFKTEEDYKAEKLLAPYKKAKTLERQVYELNKLQEKLPEKLKAEYKKKLEDTKKALDEQVKSAVTEFQNVQPTNDKMTDLQDTKYVVYESVENNESMMDTFVKHPIKTGMLNGKKYMVMETTNDDYWKDFMVEGQSVRTISKDAKNNTRTIIFPYIEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 15 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELRDAIKNPAIKDKEHSAPNSRPIDFEMKKKDGTQQFYHYASTVKPARVIFTDTKPEIELGLQSGQFWRKFEVYEGDKKLPVKLVSYDSVKDYAYIRFSVSNGTRAVKIVSSTHYNNKEEKYDYTLMEFAQPIYNSADKYKTEEDYKAEKLLAPYKKAKTLERQVYELNKLQDKLPEKLKAEYKKKLDDTKKALDDQVKSAVTEFQNVQPTNEKMTDLQDTKYVVFESVENNESVMDTFVKHPIKTGMLNGKKYVVMETTNDDYWKDFIVEGQRVRTVSKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 16 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIIDKDHSAPNSRPIDFEMKKKDGTQQFYHYASSVKPARVIFTDSGPEIELGLQSGQFWRKFEVYEGDKKLPIKLVSYDTVKDYAYIRFPVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 17 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIIDKDHSAPNSRPIDFEMKKKDGTQQFYHYASSVKPARVIFTDSGPEIELGLQSGQFWRKFEVYEGDKKLPIKLVSYDTVKDYAYIRFPVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESEENNESMMDTFVKHPIYTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 18 - protein, 645 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIIDKDHSAPNSRPIDFEMKKKDGTQQFYHYASSVKPARVIFTDSGPEIELGLQSGQFWRKFEVYEGDKKLPIKLVSYDTVKDYAYIRFPVSNGTKAVKIVSSTHFNNKEEKYDYTLMEFAQPIYNSADKFKDEEDYKAEKLLAPYKKAKTLERQVYELNKIQDKLPEKLKAEYKKKLEDTKKALDEQVKSAITEFQNVQPTNEKMTDLQDTKYVVYESEENNESMMDTFVKHPIYTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 19 - protein, 650 aa, Artificial Sequence
  ORF0657n mutant
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREGSEAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 20 - protein, 118 aa, Artificial Sequence
  2H2 Vh
DVHLVESGPGLVAPSQNLSITCTVSGFSLSRYGVHWVRQPPGKGLEWLGLIWAGGVTIYNSTLMSRLSISKDSSKSQVFLKMNSLQIDDTAIYYCAREASRDHYFDYWGQGTTLTVSS

SEQ ID NO: 21 - protein, 107 aa, Artificial Sequence
  2H2 Vl
DIVMTQSPAIMSASPGEKITMTCSASSSVSYIYWYQQKSGTSPKRWIYDTSKLASGVPFRFSGGGSGTSFSLTISSMEAEDAATYYCQQWSSNPLTFGAGTKLEIKR

SEQ ID NO: 22 - protein, 213 aa, Artificial Sequence
  Mouse 2H2B3 Variable and Human Kappa Constant Region
DIVMTQSPAIMSASPGEKITMTCSASSSVSYIYWYQQKSGTSPKRWIYDTSKLASGVPFRFSGGGSGTSFSLTISSMEAEDAATYYCQQWSSNPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 448 aa, Artificial Sequence
  Mouse 2H2B3 Variable and Human IgG1 Constant Region
DVHLVESGPGLVAPSQNLSITCTVSGFSLSRYGVHWVRQPPGKGLEWLGLIWAGGVTIYNSTLMSRLSISKDSSKSQVFLKMNSLQIDDTAIYYCAREASRDHYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - DNA, 354 bp, Artificial Sequence
  Sequence encoding 2H2 Vh
gatgtgcacctggtggagtcaggacctggcctggtggcgccctcacagaacctgtccatcacttgcactgtctctgggttctcattatcccgctatggtgtacactgggttcgccagcctccaggaaagggtctggagtggctgggactaatatgggctggtggagtcacaatttataattcgactctcatgtccagactgagcatcagcaaagacagctccaagagccaggttttcctaaaaatgaacagtctacaaattgatgacacagccatttactactgtgccagagaagcatctcgggaccactactttgactactggggccaaggcaccactctcacagtctcctcg

SEQ ID NO: 25 - DNA, 321 bp, Artificial Sequence
  Sequence encoding 2H2 Vl
gatattgtgatgacccagtctccagcaatcatgtctgcatctccaggggagaagatcaccatgacctgcagtgccagctcaagtgttagttacatctactggtaccagcagaagtcaggcacctcccccaaaagatggatttatgacacatccaaactggcttctggagtcccttttcgcttcagtggcggtgggtctgggacctctttctctctcacaatcagcagcatggaggctgaagatgctgccacttattactgccagcagtggagtagtaacccactcacgttcggtgctgggaccaagctggaaataaaacgt

SEQ ID NO: 26 - protein, 19 aa, Artificial Sequence
  Heavy chain leader sequence
MEWSWVFLFFLSVTTGVHS

SEQ ID NO: 27 - protein, 20 aa, Artificial Sequence
  Light chain leader sequence
MSVPTQVLGLLLLWLTDARC

SEQ ID NO: 28 - DNA, 36 bp, Artificial Sequence
  Oligonucleotide primer
acagatgccagatgcgatattgtgatgacccagtct

SEQ ID NO: 29 - DNA, 36 bp, Artificial Sequence
  Oligonucleotide primer
tgcagccaccgtacgttttatttccagcttggtccc

SEQ ID NO: 30 - DNA, 36 bp, Artificial Sequence
  Oligonucleotide Primer
acaggtgtccactcggatgtgcacctggtggagtca

SEQ ID NO: 31 - DNA, 36 bp, Artificial Sequence
  Oligonucleotide Primer
gcccttggtggatgccgaggagactgtgagagtggt

SEQ ID NO: 32 - protein, 20 aa, Artificial Sequence
  Linker
GGGGSGGGGSGGGGSGGGGS

SEQ ID NO: 33 - DNA, 30 bp, Artificial Sequence
  Oligonucleotide primer
gtattaggaattcggcccccgaggccgagg

SEQ ID NO: 34 - DNA, 33 bp, Artificial Sequence
  Oligonucleotide primer
gcattactcgcggcccagccggccatggcggac

(19) United States
(12) Patent Application Publication
(10) Pub. No.: US 2011/0091480 A1
(43) Pub. Date: Apr. 21, 2011

(54) Antigen-Binding Proteins Targeting S. Aureus Orf0657n

(76) Inventors: Martha J. Brown, Schwenksville, PA (US); Annaliesa S. Anderson, Upper Saddle River, NJ (US); Leslie D. Cope, Hatfield, PA (US); Kathrin Ute Jansen, Allendale, NJ (US); Tessie McNeely, Gwynedd Valley, PA (US); Barrett R. Harvey, Pearland, TX (US); Eberhard Durr, Quakertown, PA (US); Robin Ernst, Kintnersville, PA (US)

(21) Appl. No.: 12/223,110

(22) PCT Filed: Jan. 23, 2007
(86) PCT No.: PCT/US07/01687

§ 371 Date: Jul. 22, 2008

Related U.S. Application Data
(60) Provisional application No. 60/763,023, filed on Jan. 27, 2006.

(51) Int. Cl. A61K 39/40; C07K 16/12; C07H 21/04; A61P 31/04; C12N 5/10; C12P 21/04; G01N 33/567; G01N 33/566; C12N 5/16

(52) U.S. Cl. 424/165.1; 530/389.5; 530/387.3; 536/23.53; 435/325; 435/69.6; 435/7.21; 436/501; 435/326

(57) ABSTRACT

The present invention features antigen binding protein that bind an ORF0657n target region (SEQ ID NO: 1). ORF0657n is an S. aureus protein. ORF0657n target regions are provided by the mAb 1G3.BD4, mAb 2H2.BE11, mAb 13C7.BC1, and mAb 13G11.BF3 binding sites. In a lethal model challenge, mAb 2H2.BE11 and mAb 13C7.BC1 provided for increased survival against S. aureus infection. There was also protection demonstrated in an ex vivo model with either the IgG1 or the IgG2b form of mAb 2H2; and in a passive immunization murine indwelling catheter model using mAb 2H2.BE11.

### RELATED APPLICATIONS

The present application claims priority to U.S. Provisional Application No. 60/763,023, filed Jan. 27, 2006, which is hereby incorporated by reference herein.

### BACKGROUND OF THE INVENTION

The references cited throughout the present application are not admitted to be prior art to the claimed invention.

Staphylococcus aureus is a pathogen responsible for a wide range of diseases and conditions. Examples of diseases and conditions caused by S. aureus include bacteremia, infective endocarditis, folliculitis, furuncle, carbuncle, impetigo, bullous impetigo, cellulitis, botryomyosis, toxic shock syndrome, scalded skin syndrome, central nervous system infections, infective and inflammatory eye disease, osteomyelitis and other infections of joints and bones, and respiratory tract infections. (The Staphylococci in Human Disease, Crossley and Archer (eds.), Churchill Livingstone Inc. 1997.)

Immunological based strategies can be employed to control S. aureus infections and the spread of S. aureus. Immunological based strategies include passive and active immunization. Passive immunization employs immunoglobulins targeting S. aureus. Active immunization induces immune responses against S. aureus.

### SUMMARY OF THE INVENTION

The present invention features antigen binding protein that bind an ORF0657n target region (SEQ ID NO: 1). ORF0657n is an S. aureus protein. ORF0657n target regions are provided by the mAb 1G3.BD4, mAb 2H2.BE11, mAb 13C7.BC1, and mAb 13G11.BF3 binding sites. In a lethal model challenge, mAb 2H2.BE11 and mAb 13C7.BC1 provided for increased survival against S. aureus infection. There was also protection demonstrated in an ex vivo model with either the IgG1 or the IgG2b form of mAb 2H2; and in a passive immunization murine indwelling catheter model using mAb 2H2.BE11.

Mouse hybridoma cell lines producing mAb 1 G3.BD4, mAb 2H2.BE11, mAb 13C7.BC1, and mAb 13G11.BF3 were deposited with the American Type Culture Collection,'10801 University Boulevard, Manassas, Va. 20110-2209, in accordance with Budapest Treaty on Sep. 30, 2005. The cells lines were designated: ATCC No. PTA-7124 (producing mAb 2H2.BE11), ATCC No. PTA-7125 (producing mAb 13C7.BC1), ATCC No. PTA-7126 (producing mAb 1G3.BD4), and ATCC No. PTA-7127 (producing mAb 13G11.BF3).

Thus, a first aspect of the present invention features an isolated antigen binding protein comprising a first variable region and a second variable region. The first and second variable regions bind one or more target regions selected from the group consisting of: mAb 1G3.BD4 target region, mAb 2H2.BE11 target region, mAb 13C7.BC1 target region, and mAb 13G11.BF3 target region.

Reference to “isolated” indicates a different form than found in nature. The different form can be, for example, a different purity than found in nature and/or a structure that is not found in nature. A structure not found in nature includes recombinant structures where different regions are combined together, for example, humanized antibodies where one or more murine complementary determining regions is inserted onto a human framework scaffold or a murine antibody is resurfaced to resemble the surface residues of a human antibody, hybrid antibodies where one or more complementary determining regions from an antigen binding protein is inserted into a different framework scaffold, and antibodies derived from natural human sequences where genes coding for light and heavy variable domains were randomly combined together.

The isolated protein is preferably substantially free of serum proteins. A protein substantially free of serum proteins is present in an environment lacking most or all serum proteins.

A “variable region” has the structure of an antibody variable region from a heavy or light chain. Antibody heavy and light chain variable regions contain three complementary determining regions interspaced onto a framework. The complementary determining regions are primarily responsible for recognizing a particular epitope.

A target region is defined with respect to the ORF0657n region (SEQ ID NO: 1) bound by mAb 1G3.BD4, mAb 2H2.BE11, mAb 13C7.BC1, or mAb 13G11.BF3. For example, the mAb 1G3.BD4 target region is the ORF0657n region to which mAb 1G3.BD4 binds.

A protein binding an identified target region competes with either mAb 1G3.BD4, mAb 2H2.BE11, mAb 13C7.BC1, or mAb 13G11.BF3 for binding to the target region. For example, a protein competing with mAb 1G3.BD4 binding to ORF0657n binds to the mAb 1G3.BD4 target region.

A protein that competes with either the monoclonal antibody mAb 1G3.B3, mAb 2H2.B8, mAb 13C7.D12, or mAb 13G11.C11 reduces binding of the monoclonal antibody to ORF0657n by at least about 20%, preferably at least about 50%, when excess and equal amounts of the competing protein and monoclonal antibody are employed.

Reference to “protein” indicates a contiguous amino acid sequence and does not provide a minimum or maximum size limitation. One or more amino acids present in the protein may contain a post-translational modification, such as glycosylation or disulfide bond formation.

A preferred antigen binding protein is a monoclonal antibody. Reference to a “monoclonal antibody” indicates a collection of antibodies having the same, or substantially the same, complementary determining region, and binding specificity. The variation in the antibodies is that which would occur if the antibodies were produced from the same construct(s).

Monoclonal antibodies can be produced, for example, from a particular hybridoma and from a recombinant cell containing one or more recombinant genes encoding the antibody. The antibody may be encoded by more than one recombinant gene where, for example, one gene encodes the heavy chain and one gene encodes the light chain.

Another aspect of the present invention describes a nucleic acid containing a recombinant gene comprising a nucleotide sequence encoding an antibody variable region. The antibody variable region can bind a target region selected from the group consisting of: mAb IG3.BD4 target region, mAb 2H2.BE11 target region, mAb 13C7.BC1, and mAb 13G11.BF3 target region.

A recombinant gene contains recombinant nucleic acid encoding a protein along with regulatory elements for proper transcription and processing (which may include translational and post translational elements). The recombinantnucleic acid by virtue of its sequence and/or form does not occur in nature. Examples of recombinant nucleic acid include purified nucleic acid, two or more nucleic acid regions combined together providing a different nucleic acid than found in nature, and the absence of one or more nucleic acid regions (e.g., upstream or downstream regions) that are naturally associated with each other.

Another aspect of the present invention describes a recombinant cell comprising one or more recombinant genes encoding an antibody variable region that binds to a target region selected from the group consisting of: mAb IG3.BD4 target region, mAb 2H2.BE11 target region, mAb 13C7.BC1, and mAb 13G11.BF3 target region. Multiple recombinant genes are useful, for example, where one gene encodes an antibody heavy chain or fragment thereof containing the Vₕ region and another nucleic acid encodes an antibody light chain or fragment thereof containing the V₁ region.

Another aspect of the present invention comprises a method of producing a protein comprising an antibody variable region. The method comprising the steps of: (a) growing a recombinant cell comprising recombinant nucleotide acid encoding for a protein under conditions wherein the protein is expressed; and (b) purifying the protein.

Another aspect of the present invention describes a pharmaceutical composition. The composition contains a therapeutically effective amount of an antigen binding protein and a pharmaceutically acceptable carrier.

A therapeutically effective amount is an amount sufficient to provide a useful therapeutic or prophylactic effect. For a patient infected with S. aureus, an effective amount is sufficient to achieve one or more of the following effects: reduce the ability of S. aureus to propagate in the patient or reduce the amount of S. aureus in the patient. For a patient not infected with S. aureus, an effective amount is sufficient to achieve one or more of the following: a reduced susceptibility to S. aureus infection or a reduced ability of the infecting bacterium to establish persistent infection for chronic disease.

Another aspect of the present invention describes a method of detecting the presence of an OFR0657n antigen in a solution or on a cell. The method involves providing a binding protein described herein to the solution or cell and measuring the ability of the binding protein to bind to the antigen in the solution or cell. Measurements can be quantitative or qualitative.

Reference to ORF0657n antigen includes full-length ORF0657n or a derivative thereof having an epitope that is recognized by mAb 1G3.B3, mAb 2H2.B8, mAb 13C7.D12, or mAb 13G11.C11. Examples of derivatives include truncated versions; and full-length or truncated versions of ORF0657n containing one or more of the following amino acid alterations: one or more additions, one or more substitutions, and one or more deletions.

Another aspect of the present invention features a method of treating a patient against a S. aureus infection. The method comprises the step of administering to the patient an effective amount of an antigen binding protein described herein. The patient being treated may, or may not, be infected with S. aureus. Preferably, the patient is a human.

Another aspect of the present invention describes a cell line producing a protein that is either mAb 1G3.B3, mAb 2H2.B8, mAb 13C7.D12, or mAb 13G11.C11, or that competes with either mAb IG3.B3, mAb 2H2.B8, mAb 13C7.D12, or mAb 13G11.C11 for binding to ORF0657n. Preferred cells lines are hybridomas, and recombinant cell lines containing recombinant nucleic acid encoding the protein.

Reference to open-ended terms such as “comprises” allows for additional elements or steps. Occasionally phrases such as “one or more” are used with or without open-ended terms to highlight the possibility of additional elements or steps.

Unless explicitly stated reference to terms such as “a” or “an” is not limited to one. For example, “a cell” does not exclude “cells”. Occasionally phrases such as one or more are used to highlight the possible presence of a plurality.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1 illustrates the structure of an IgG molecule. “V_{L}” refers to a light chain variable region. “V_{H}” refers to a heavy chain variable region. “C_{L}” refers to a light chain constant region. “CH₁”, “CH₂” and “CH₃” are heavy chain constant regions. Dashed lines indicate disulfide bonds.

FIG. 2 illustrates a matrix outlining the reactivities of different monoclonal antibodies in a pair-wise binding study. The panel of monoclonal antibodies fell into three reactive areas by the BIACORE® method.

FIGS. 3A-3C: Groups of BALB/c mice (n=20) were treated 20 hours prior to bacterial challenge with an i.p. injection of: ▪, mAb 13C7.BC1; □, mAb 6G6.A8 (isotype control); or O, PBS. Mice were challenged with S. aureus by i.v. injection and survival was monitored. FIG. 3A-0.49 mg mAb 13C7.BC1; 0.45 mg mAb 6G6.A8; and 9.8×10⁸ CFU S. aureus Becker. FIG. 3B- 0.49 mg mAb 13C7.BC1; 0.45 mg mAb 6G6.A8; and 9.6×10⁸ CFU S. aureus Becker. FIG. 3C- 0.50 mg mAb13C7.BC1; 0.45 mg mAb 6G6; and 9.9×10⁸ CFU S. aureus Becker.

FIGS. 4A and 4B: Groups of BALB/c mice (n=20) were treated 20 hours prior to bacterial challenge with an i.p. injection of: ▪, mAb 13C7.BC1 (0.5 mg); □, mAb 6G6.A8 (isotype control) (0.5 mg); or 0, PBS (0.5 ml). Mice were challenged with S. aureus by i.v. injection and survival was monitored. FIG. 4A illustrates results with 2.09×10⁸ CFU S. aureus UK58. FIG. 4B illustrates results with 2.15×10⁸ S. aureus UK 58.

FIGS. 5A-5C: Groups of BALB/c mice (n =20) were treated 20 hours prior to bacterial challenge with an i.p. injection of: ▪, mAb 2H2.BE11, □, mAb 6G6.A8 (isotype control); O, PBS. Mice were challenged with S. aureus by i.v. injection and survival was monitored. FIG. 5A- 0.43 mg mAb 2H2.BE11; 0.5 mg mAb 6G6.A8; and 9.8×10⁸ CFU S. aureus Becker. FIG. 5B- 0.43 mg mAb 2H2.BE11; 0.5 mg mAb 6G6.A8; and 8.3×10⁸ CFU S. aureus Becker. FIG. 5C- 0.43 mg mAb 2H2.BE11; 0.5 mg mAb 6G6.A8; and 9.3×10⁸ CFU S. aureus Becker.

### DETAILED DESCRIPTION OF THE INVENTION

ORF0657n is an S. aureus protein located at the S. aureus outer membrane. ORF0657n has been found to be well conserved in different strains of S. aureus. (Anderson et al., International Publication No. WO 2005/009379, International Publication Date Feb. 3, 2005.) Different ORF0657n derivatives can be used to produce a protective immune response against S. aureus infection. (Anderson et al., International Publication No. WO 2005/009379, International Publication Date Feb. 3, 2005.)

Due to their ability to recognize ORF0657n, the antigen binding proteins described herein can be used, for example, as a tool in the production, characterization, or study of ORF0657n based antigens. Antigen binding protein recognizing appropriate ORF0657n epitopes can also be used agent to treat S. aureus infection.

### I. Antigen Binding Protein

Antigen binding proteins contain an antibody variable region providing for specific binding to an epitope. The antibody variable region can be present in, for example, a complete antibody, an antibody fragment, and a recombinant derivative of an antibody or antibody fragment.

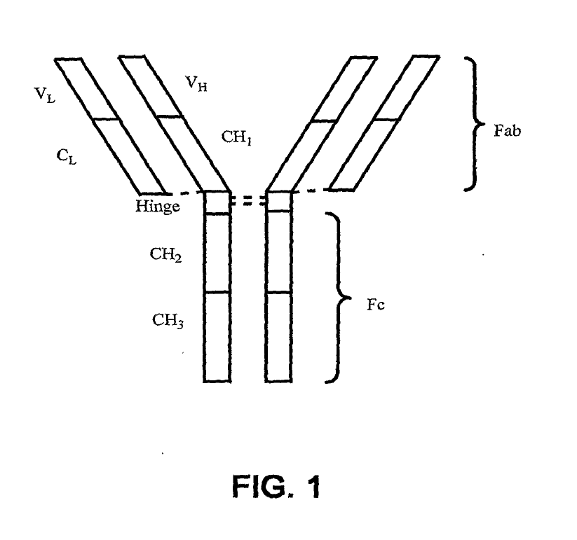

Different classes of antibodies have different structures. Different antibody regions can be illustrated by reference to IgG (FIG. 1). An IgG molecule contains four amino acid chains: two longer length heavy chains and two shorter light chains. The heavy and light chains each contain a constant region and a variable region. Within the variable regions are three hypervariable regions responsible for antigen specificity. (See, for example, Breitling et aL, Recombinant Antibodies, John

Wiley & Sons, Inc. and Spektrum Akademischer Verlag, 1999; and Lewin, Genes IV, Oxford University Press and Cell Press; 1990.)

The hypervariable regions (also referred to as complementarity determining regions), are interposed between more conserved flanking regions (also referred to as framework regions). Amino acids associated with framework regions and complementarity determining regions can be numbered and aligned as described by Kabat et al., Sequences of Proteins of Immunological Interest, U.S. Department of Health and Human Services, 1991.

The two heavy chain carboxyl regions are constant regions joined by disulfide binding to produce an Fe region. The Fc region is important for providing antibody biological activity such as complement and macrophage activation. Each of the two heavy chains making up the Fc region extend into different Fab regions through a hinge region.

In higher vertebrates there are two classes of light chains and five classes of heavy chains. The light chains are either x or λ. The heavy chains define the antibody class and are either α, δ, ε, γ, or μ. For example, IgG has a γ heavy chain. Subclasses also exist for different types of heavy chains such as human γ₁, γ₂, γ₃, and γ₄. Heavy chains impart a distinctive conformation to hinge and tail regions. (Lewin, Genes IV, Oxford University Press and Cell Press, 1990.)

Antibody fragments containing an antibody variable region include Fv, Fab, and Fab₂ regions. Each Fab region contains a light chain made up of a variable region and a constant region, and a heavy chain region containing a variable region and a constant region. A light chain is joined to a heavy chain by disulfide bonding through constant regions. The light and heavy chain variable regions of a Fab region provide for an Fv region that participates in antigen binding.

The antibody variable region can be present in a recombinant derivative. Examples of recombinant derivatives include single-chain antibodies, diabody, triabody, tetrabody, and miniantibody. (Kipriyanov et al, Molecular Biotechnology 26:39-60, 2004.)

The antigen binding protein can contain one or more variable regions recognizing the same or different epitopes. (Kipriyanov et al., Molecular Biotechnology 26:39-60, 2004.)

### II. Generation of Antigen Binding Protein Directed to an Identified Target Region

Different antigen binding proteins directed to the mAb 1G3.BD4 target region, mAb 2H2.BE11 target region, mAb 13C7.BC1 target region, or mAb 13G11.BF3 target region can be generated starting with the respective monoclonal antibody. Alternatively, the epitope recognized by a binding protein can be used to select additional binding proteins.

The mAb 2H2.BE11 target region appears to be located at approximately amino acids 76-357 of ORF0657n. A polypeptide containing amino acids 76-357 of ORF0657n, or a full-length ORF0657n, can be used as a target antigen to select for antibodies. The target region of the generated antibodies can be determined.

A variety of techniques are available to select for a protein recognizing an antigen. Examples of such techniques include use of phage display technology and hybridoma production. Human antibodies can be produced using chimeric mice such as a XenoMouse or Trans-Chromo mouse. (E.g., Azzazy et al., Clinical Biochemistry 35:425-445, 2002, Berger et al., Am. J. Med. Sci. 324(1):14-40, 2002.)

The monoclonal antibodies mAb 1G3.BD4, mAb 2H2.BE11, mAb 13C7.BC1, and mAb 13G11.BF3 contain variable regions recognizing ORF0675n. Additional binding proteins recognizing ORF0657n can be produced based on antibody variable regions. Additional binding proteins can, for example, be produced by modifying an existing monoclonal antibody and by using variable region sequence information. Protein construction and sequence manipulation can be performed using recombinant nucleic acid techniques.

The monoclonal antibodies mAb 1G3.BD4, mAb 2H2.BE11, mAb 13C7.BC1, and mAb 13G11.BF3 are murine antibodies. For human therapeutic applications, preferred binding proteins based on such mAb's are designed to reduce the potential generation of human anti-mouse antibodies recognizing the murine regions.

The potential generation of human anti-mouse antibodies can be reduced using techniques such as murine antibody humanization, de-immunization, and chimeric antibody production. (See, for example, O′Brien et al., Humanization of Monoclonal Antibodies by CDR Grafting, p 81-100, From Methods in Molecular Biology Vol. 207: Recombinant antibodies for Cancer Therapy: Methods and Protocols (Eds. Welschof and Krauss) Humana Press, Totowa, New Jersey, 2003; Kipriyanov et al., Molecular Biotechnology 26:39-60, 2004; Gonzales et al., Tumor Biol. 26:31-43, 2005, Presta, Advanced Drug Delivery Reviews 58:640-656, 2006, Tsurushita et al., Methods 36:69-83, 2005, Roque et al., Biotechnol. Prog. 20:639-654, 2004.)

Murine antibodies can be humanized using techniques such as grafting complementary determining regions into a framework region or resurfacing. Resurfacing (also known as veneering) involves modifying a variable region so the surface exposed regions are humanized.

Grafting complementary determining regions involves taking such regions or a portion of such regions from, for example, a murine source and inserting the regions into a human variable region framework. The human framework used for grafting can be selected based on sequence homology to the variable region (e.g., murine) from which the region was obtained. Essential framework residues associated with grafted complementary determining regions should also be provided in the new framework.

De-immunization involves altering potential linear T-cell epitopes present in the antibody. The epitopes can be identified based on a bioinformatics scan of know human HLA class I and/or class II epitopes. (Presta, Advanced Drug Delivery Reviews 58:640-656, 2006.)

A chimeric antibody contains a human constant region along with a variable region from a different organism, such as a mouse. The human constant region provides an Fc region.

Additional examples of alterations include providing a variable region in, for example, a single chain antibody, a diabody, a triabody, a tetrabody, and a miniantibody. (Kipriyanov et aL, Molecular Biotechnology 26:39-60, 2004.) The antigen binding protein can contain one or more variable regions recognizing the same or different epitopes. (Id.) Additional embodiments of the present invention are directed to a single chain antibody, a diabody, a triabody, a tetrabody, or a miniantibody directed to the mAb 1G3.BD4, mAb 2H2.BE11, mAb 13C7.BC1, or mAb 13G11.BF3 binding site.

### III. Binding Protein Directed to the mAb 2H2.BE11 Target Region

As described in the Examples provided below, the mAb 2H2.BE11 target region was further characterized and the amino acids sequence of the variable regions was determined. The identified target region and the sequence information facilitate obtaining different binding proteins directed to the mAb 2H2.BE11 target region.

In an embodiment of the present invention, the binding protein binds to a polypeptide consisting of amino acids 76-357 of SEQ ID NO: 1. Preferably, the binding protein is either a human antibody, a humanized antibody, a de-immunized antibody, or chimeric antibody. Preferred antibodies are isolated antibodies and monoclonal antibodies.

The amino acids sequences of the mAb 2H2.BE11 variable regions are provided by SEQ ID NO: 20 (Vₕ) and SEQ ID NO: 21 (V₁). The complementary determining regions (CDR's) within Vₕ were identified at amino acids 36-45, 50-65, and 98-107. The CDR's within V₁ were identified at amino acids 24-33, 49-55, and 88-96 of SEQ ID NO: 21.

In different embodiments directed to a Vₕ region, the binding protein binds the mAb 2H2.BE11 target region and comprises, consists, or consists essentially of: a first Vₕ CDR comprising, consisting, or consisting essentially of amino acids 36-45 of SEQ ID NO: 20 or a sequence differing from amino acids 36-45 by one amino acid; a second Vₕ CDR comprising, consisting, or consisting essentially of amino acids 50-65 of SEQ ID NO: 20 or a sequence differing from amino acids 50-65 by one amino acid; and a third Vₕ CDR comprising, consisting, or consisting essentially of amino acids 98-107 of SEQ ID NO: 20 or a sequence differing from amino acids 98-107 by one amino acid.

In different embodiments directed to a VI region, the binding protein binds the mAb 2H2.BE11 target region and comprises, consists, or consists essentially of a first V₁ CDR comprising, consisting, or consisting essentially of amino acids 24-33 of SEQ ID NO: 21 or a sequence differing from amino acids 24-33 by one amino acid; a second V₁ CDR comprising, consisting, or consisting essentially of amino acids 49-55 of SEQ ID NO: 21 or a sequence differing from amino acids 49-55 by one amino acid; and a third V₁ CDR comprising, consisting, or consisting essentially of amino acids 88-96 of SEQ ID NO: 21 or a sequence differing from amino acids 88-96 by one amino acid.

Reference to “consisting essentially of” with respect to a variable region, CDR region, or antibody sequence, indicates the possible presence of one or more additional amino acids, where such amino acids do not significantly decrease binding to the target.

An amino acid difference can be an amino acid deletion, insertion, or substitution. In substituting amino acids to maintain activity, the substituted amino acids should have one or more similar properties such as approximately the same charge, size, polarity and/or hydrophobicity.

Preferably, an amino acid substitution is a conservative substitution. A conservative substitution replaces an amino acid with another amino acid having similar properties. Table 1 provides a list of groups of amino acids, where one member of the group is a conservative substitution for another member.

TABLE 1
Conservative Substitutions

Ala, Val, Ile, Leu, Met
Ser, Thr
Tyr, Trp
Asn, Gln
Asp, Glu
Lys, Arg, His

In additional embodiments the Vₕ region is either SEQ ID NO: 20, a humanized SEQ ID NO: 20, or a de-immunized SEQ ID NO: 20; and/or the V₁ region is either SEQ ID NO: 21, a humanized. SEQ ID NO: 21, or a de-immunized SEQ ID NO: 21.

In different embodiments focusing on an antibody, the antibody comprises, consists, or consists essentially of: (a) a heavy chain comprising a Vₕ region as described in this Section III, and a human hinge, CH₁, CH₂, and CH₃ regions from an IgG₁, IgG₂, IgG₃ or IgG₄, and (b) a light chain comprising a V₁ region as described above in this section III, and a human kappa C_{L} or human lambda C_{L}. In further embodiments: the antibody comprises, consists, or consists essentially of: (a) a heavy chain comprising a Vₕ region as described in this Section III, and a human hinge, CH₁, CH₂, and CH₃ regions from an IgG₁ or IgG₂ and (b) a light chain comprising a V₁ region as described above in this Section III, and a human kappa C_{L}; and the heavy chain consists essentially of the amino acid sequence of SEQ ID NO: 22 and/or the light chain consists essentially of the amino acid sequence of SEQ ID NO: 23.

In additional embodiments the antigen-binding protein described herein has Vₕ and V₁ regions providing an affinity K_{D} at least about 100 nM, preferably at least about 30 nM to the target antigen. Binding to the target antigen can be determined as described in Example 11, using an ORF0657n fragment from amino acids 42-486

Preferred binding proteins for the different embodiments are an antibody. More preferably the antibody is isolated or a monoclonal antibody.

### IV. Protein Production

Antigen binding protein are preferably produced using recombinant nucleic acid techniques or through the use of a hybridoma. Recombinant nucleic acid techniques involve constructing a nucleic acid template for protein synthesis. Hybridoma techniques involve using an immortalized cell line to produce the antigen binding protein. Suitable recombinant nucleic acid and hybridoma techniques are well known in the art. (See for example, Ausubel, Current Protocols in Molecular Biology, John Wiley, 2005, Harlow et al., Antibodies, A Laboratory Manual, Cold Spring Harbor Laboratory, 1988.)

Recombinant nucleic acid encoding an antigen binding protein can be expressed in a host cell that in effect serves as a factory for the encoded protein. The recombinant nucleic acid can provide a recombinant gene encoding the antigen binding protein that exists autonomously from a host cell genome or as part of the host cell genome.

A recombinant gene contains nucleic acid encoding a protein along with regulatory elements for protein expression. Generally, the regulatory elements that are present in a recombinant gene include a transcriptional promoter, a ribosome binding site, a terminator, and an optionally present operator. A preferred element for processing in eukaryotic cells is a polyadenylation signal. Antibody associated introns may also be present. Examples of expression cassettes for antibody or antibody fragment production are well known in art. (E.g., Persic et al., Gene 187:9-18, 1997, Boel et al., J. Immunol. Methods 239:153-166, 2000, Liang et al., J. Immunol. Methods 247:119-130, 2001, Tsurushita et al., Methods 36:69-83, 2005.)

Due to the degeneracy of the genetic code, a large number of different encoding nucleic acid sequences can be used to code for a particular protein. The degeneracy of the genetic code arises because almost all amino acids are encoded by different combinations of nucleotide triplets or “codons”. Amino acids are encoded by codons as follows:
A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons UUC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAU

Expression of a recombinant gene in a cell is facilitated using an expression vector. Preferably, the expression vector, in addition to a recombinant gene, also contains an origin of replication for autonomous replication in a host cell, a selectable marker, a limited number of useful restriction enzyme sites, and a potential for high copy number. Examples of expression vectors for antibody and antibody fragment production are well known in art. (E.g., Persic et al., Gene 187:9-18, 1997, Boel et aL, J. Immunol. Methods 239:153-166, 2000, Liang et al., J. ImmunoL Methods 247:119-130, 2001, Tsurushita at al., Methods 36:69-83, 2005.)

If desired, nucleic acid encoding an antibody may be integrated into the host chromosome using techniques well known in the art. (E.g., Ausubel, Current Protocols in Molecular Biology, John Wiley, 2005, Marks et aL, International Application Number WO 95/17516, International Publication Date Jun. 29, 1995.)

A variety of different cell lines can be used for recombinant antigen binding protein expression, including those from prokaryotic organisms (e.g., E. coli, Bacillus sp, and Streptomyces sp. (or streptomycete) and from eukaryotic (e.g., yeast, Baculovirus, and mammalian). (Breitling at al., Recombinant Antibodies, John Wiley & Sons, Inc. and Spektrum Akademischer Verlag, 1999, Kipriyanov et al., Molecular Biotechnology 26:39-60, 2004, Tsurushita et al., Methods 36:69-83, 2005.)

Preferred hosts for recombinant antigen binding protein expression provide for mammalian post translational modifications. Post translational modifications chemical modification such as glycosylation and disulfide bond formation. Another type of post translational modification is signal peptide cleavage.

Proper glycosylation can be important for antibody function. (Yoo et al., Journal of Immunological Methods 261:1-20, 2002, Li at al., Nature Biotechnology 24(2):210-215, 2006.) Naturally occurring antibodies contain at least one N-linked carbohydrate attached to a heavy chain. (Yoo at al., Journal of Immunological Methods 261:1-20, 2002.) Additional N-linked carbohydrates and O-linked carbohydrates may be present and may be important for antibody function. (Id.)

Different types of host cells can be used to provide for efficient post-translational modifications including mammalian host cells and non-mammalian cells. Examples of mammalian host cells include but are not limited to Chinese hamster ovary (Cho), HeLa, C6, PC 12, Human Embryonic Kidney (HEK293) and myeloma cells. (Yoo at al., Journal of Immunological Methods 261:1-20, 2002, Persic et al., Gene 187:9-18, 1997.) Non-mammalian cells can be modified to replicate human glycosylation. (Li at al., Nature Biotechnology 24(2):210-215, 2006.) Glycoenginnered Pichia pastoris is an example of such a modified non-mammalian cell. (Li et al., Nature Biotechnology 24(2):210-215, 2006.)

Preferred recombinant genes comprise a nucleotide sequence encoding an antibody variable region that binds to a target region selected from the group consisting of mAb IG3.BD4 target region, mAb 2H2.BE11 target region, mAb 13C7.BC1, and mAb 13G11.BF3 target region. A particular recombinant gene can encode for a protein containing one variable region or both a Vₕ and V₁ region. The recombinant gene can also encode for antibody constant regions and hinge region. If desired, an antibody can be produced using a combination of recombinant genes, where one gene encodes for a light chain and the second gene encodes for a heavy chain.

Different embodiments are provided by the nucleic acid encoding a protein described in Section II or III supra. Examples of such embodiments are provided below.

In an embodiment directed to a Vₕ encoding region, the nucleotide sequence encodes a variable region comprising, consisting, or consisting essentially of: a first Vₕ CDR comprising, consisting, or consisting essentially of amino acids 36-45 of SEQ ID NO: 20 or a sequence differing from amino acids 36-45 by one amino acid; a second Vₕ CDR comprising, consisting, or consisting essentially of amino acids 50-65 of SEQ ID NO: 20 or a sequence differing from amino acids 50-65 by one amino acid; and a third Vₕ CDR comprising, consisting, or consisting essentially of amino acids 98-107 of SEQ ID NO: 20 or a sequence differing from amino acids 98-107 by one amino acid.

In an embodiment directed to a V₁ encoding region, the nucleotide sequence encodes a variable region comprising, consisting, or consisting essentially of a first VI CDR comprising, consisting, or consisting essentially of amino acids 24-33 of SEQ ID NO: 21 or a sequence differing from amino acids 24-33 by one amino acid; a second V₁ CDR comprising, consisting, or consisting essentially of amino acids 49-55 of SEQ ID NO: 21 or a sequence differing from amino acids 49-55 by one amino acid; and a third V₁ CDR comprising, consisting, or consisting essentially of amino acids 88-96 of SEQ ID NO: 21 or a sequence differing from amino acids 88-96 by one amino acid.

In additional embodiments: the Vₕ region is either SEQ ID NO: 20, a humanized SEQ ID NO: 20, or a de-immunized SEQ ID NO: 20; and the V₁ region is either SEQ ID NO: 21, a humanized SEQ ID NO: 21, or a de-immunized SEQ ID NO: 21.

In different embodiments focusing on an antibody heavy and/or light chain, the recombinant gene encodes either or both a protein comprising, consisting, or consisting essentially of: (a) a heavy chain comprising a Vₕ region as provided in Section DI supra, a human hinge, CH₁, CH₂, and CH₃ from an IgG1, IgG2, IgG3 or IgG4 subtype or (b) a light chain comprising a V₁ region as provided in Section III supra, and a human kappa C_{L} or lambda C_{L}. In a further embodiment the heavy chain consists essentially of the amino acid sequence of SEQ ID NO: 22; and the light chain consists essentially of the amino acid sequence of SEQ ID NO: 23.

### V. Applications of Antigen Binding Proteins

Antigens containing certain ORF0657n regions can be used to provide a protective immune response against S. aureus infection. (Anderson et al., International Publication No. WO 2005/009379, International Publication Date Feb. 3, 2005.) An antigen binding protein recognizing an ORF0657n target region can be used to facilitate the production, characterization, or study of ORF0657n antigens and vaccines. Antigen binding protein recognizing appropriate epitopes can also have therapeutic applications.

Examples of different uses in the production, characterization, or study of ORF0657n related antigens and vaccines include:

1) Identifying the presence of an ORF0657n antigen, for example, by Western blot;

2) Identifying the presence of an ORF0657n antigen on a cell surface, for example, by flow cytometry. This is useful, for example, in determining expression on multiple strains of S. aureus as well as confirmation of knock-out mutants;

3) Passive protection experiments. The antibodies can be used in a lethal model to determine if a specific area of the ORF0657n protein confers protection;

4) An immunoassay. The assay can be used to monitor antigen quality, product production and stability;

5) As a control in mouse potency assays to monitor immunogenicity of an antigen vaccine product; and

6) Serology assays can utilize a monoclonal antibody in a competitive format to identify an immune response to ORF0657n derived antigen vaccinated patients.

Techniques for using antigen binding proteins, such as monoclonal antibodies, in the production, characterization, or study of a target protein are well known in the art. (See, for example,

Ausubel, Current Protocols in Molecular Biology, John Wiley, 2005, Harlow et al., Antibodies, A Laboratory Manual, Cold Spring Harbor Laboratory, 1988, Harlow et al., Using Antibodies, Cold Spring Harbor Laboratory, Cold Spring Harbor, N.Y., Cold Spring Harbor Laboratory Press, 1999, Lipman et al., ILAR Journal 46:258-268, 2005.)

In an embodiment of the present invention, the presence of an ORF0657n antigen in a solution, bound to a microsphere or on a cell is determined using an antigen binding protein. The ability of the binding protein to bind to a protein present in the solution or cell can be determined using different techniques such as a Western blot, enzyme-linked immunosorbent assay (ELISA), flow cytometry, and Luminex immunoassay.

### VI. Treatment

Therapeutic and prophylactic treatment can be performed on a patient using an antigen binding protein binding to an appropriate target region. Therapeutic treatment is performed on those persons infected with S. aureus. Prophylactic treatment can be performed on the general population or a subset of the general population. A preferred subset of the general population are those persons at an increased risk of S. aureus infection.

A “patient” refers to a mammal capable of being infected with S. aureus. Preferably, the patient is a human. However, other types of mammals such as cows, pigs, sheep, goats, rabbits, horses, dogs, cats, monkeys, rats, and mice, can be infected with S. aureus. Treatment of non-human patients is useful in protecting pets and livestock, and in evaluating the efficacy of a particular treatment.

Persons with an increased risk of S. aureus infection include health care workers; hospital patients; patients with a weakened immune system; patients undergoing surgery; patients receiving foreign body implants, such as a catheter or a vascular device; patients facing therapy leading to a weakened immunity; and persons in professions having an increased risk of burn or wound injury. (The Staphylococci in Human Disease, Crossley and Archer (ed.), Churchill Livingstone Inc. 1997.)

In an embodiment, a patient is administered an antigen binding protein in conjunction with surgery or a foreign body implant. Reference to “surgery or a foreign body implant” includes surgery with or without providing a foreign implant, and providing a foreign implant with or without surgery. The timing of administration can be designed to achieve prophylactic treatment and/or therapeutic treatment. Administration is preferably started around the same time as surgery or implantation.

Guidelines for pharmaceutical administration in general are provided in, for example, Remington's Pharmaceutical Sciences 20^{th} Edition, Ed. Gennaro, Mack Publishing, 2000; and Modern Pharmaceutics 2^{nd} Edition, Eds. Banker and Rhodes, Marcel Dekker, Inc., 1990.

Pharmaceutically acceptable carriers facilitate storage or administration of an antigen binding protein. Substances used to stabilize protein solution formulations include carbohydrates, amino acids, and buffering salts. (Middaugh et al., Handbook of Experimental Pharmacology 137:33-58, 1999.)

Antigen binding proteins can be administered by different routes such as intraveneous, subcutaneous, intramuscular, or mucosal. Subcutaneous and intramuscular administration can be performed using, for example, needles or jet-injectors. Mucosal delivery, such as nasal delivery, can involve using enhancers or mucoadhesives to produce a longer retention time at adsorption sites. (Middaugh et al., Handbook of Experimental Pharmacology 137:33-58, 1999.)

Suitable dosing regimens are preferably determined taking into account factors well known in the art including age, weight, sex and medical condition of the patient; the route of administration; the desired effect; and the particular compound employed. It is expected that an effective dose range should be about 0.1 mg/kg to 20 mg/kg, or 0.5 mg/kg to 5 mg/kg. The dosing frequency can vary depending upon the effectiveness and stability of the compound. Examples of dosing frequencies include biweekly, weekly, monthly and bimonthly.

### VII. EXAMPLES

Examples are provided below further illustrating different features of the present invention. The examples also illustrate useful methodology for practicing the invention. These examples do not limit the claimed invention.

### Example 1:

### Generation of Monoclonal Antibodies to ORF0657n

Monoclonal antibodies directed to ORF0657n (SEQ ID NO: 1) were generated using ORF0657n-C/e (SEQ ID NO: 2) or ORF0657n-H/y (SEQ ID NO: 3) as an antigen. The antibodies were identified and characterized by ELISA and flow cytometry.

Mice and Immunizations: Female BALB/c mice, 4-5 weeks old, were purchased from Taconic (Germantown, N. Y.). Mice were immunized intramuscularly (i.m.) on days 0, 7, and 21, with 20 μg of E. coli produced ORF0657n-C/e antigen or Yeast expressed ORF0657n-H/y antigen, formulated on aluminum hydroxyphosphate adjuvant. (Anderson et al., international Publication No. WO 2005/009379, international Publication Date Feb. 3, 2005.) A final intravenous injection (i.v.) of 20 μg of protein in phosphate buffered saline (PBS) was given to mice three days prior to the fusion. Mice were sacrificed and the spleens removed for cell fusion.

MAb Production: Lymphocytes prepared from spleens were fused with the mouse myeloma partner SP2/0-Ag14 (ATCC 1581) by polyethylene glycol 1500 (Boehringer Mannheim) at a ratio of 3:1. The fusions were plated into 96-well flat-bottomed microtiter plates in Dulbecco's Modification of Eagle's Medium, high glucose, pyruvate (DMEM) containing 20% fetal bovine serum, hypoxanthine (10⁻⁴ M), thymidine (10⁻⁵M), Aminopterin (4×10⁻⁷ M) was added 24 hours later. Supernatants from growing hybridomas were screened by ELISA for reactivity to ORF0657n as described below. Positive wells were cloned by limiting dilution and retested for ELISA reactivity. Monoclonal antibodies were classified with an antibody-isotyping kit (Roche Diagnostics Corporation, Indianapolis, Ind).

ELISA: Costar medium binding microtiter plates were coated overnight at 2-8° C. with 50 nanograms per well of E. coli expressed SEQ ID NO: 2 in PBS. The plate was washed three times with PBS, 0.05% Tween20 and blocked with 1% Bovine serum albumin, PBS, 0.05% Tween20 (assay diluent) for at least 1 hour. The plate was washed as before and supernatants from the fusion wells or cloned hybridomas were added and allowed to incubate for 2 hours at room temperature. The plate was washed as before and a Goat anti-mouse IgG (H+L)-HRP conjugate (Zymed) (1:8000 in assay diluent) added and . allowed to incubate for 1 hour at room temperature. Assay plates were developed with TMB substrate, the reaction stopped with 2.0 N H₂SO₄ and read in a plate reader at OD 450 nm. Wells were considered positive that had an optical density at 450 nm of >1.0.

Flow Cytometry: Prepared glycerol stocks of S. aureus passaged under iron-starved conditions (in RPMI) were used to evaluate mAb for ORF0657n binding. Frozen glycerol stock cells were thawed and resuspended in PBS; 1% bovine serum albumin; 0.1% sodium azide, 0.2% Pig IgG (Sigma) (PAAG) to a concentration of 5×10⁷ CFU/50 ul. A 50 ul aliquot of the cells were placed in a 1.5 ml Eppendorf tube per reaction. Fifty microliters of the hybridoma culture were added to each reaction tube and incubated for 1 hour at room temperature. The cells were washed by adding 1 mL of phosphate buffered saline; 1% bovine serum albumin; 0.1% sodium azide (PAA) to the tube. The cells were pelleted by centrifugation (5500 rpm, 5 minutes). The supernatant was removed and the cells were mixed with 100 μL of secondary antibody (FITC-labeled goat anti-mouse Ig (BD Pharmingen) diluted 1:100 in PAAG). Incubation was for 1 hour at room temperature in the dark. After incubation, 1 mL PAA was added to the reaction mixture, the cells were pelleted (5500 rpm, 5 minutes) and supernatant removed. The pellets were resuspended in 1 mL of PBS and transferred to 12×75 mm tubes for FAC analysis.

Tubes were run on a BD-FACSCalibur flow cytometer instrument gated for bacterial cells and measuring the amount of FITC associated with the cells. A standard antibody with known binding to the surface of S. aureus was run in every assay. A negative control was run as cells and the secondary conjugate alone. Hybridoma wells were considered positive if the geometric mean value was greater than 30.

Two separate fusions resulted in a panel of twelve monoclonal antibodies (mAb). All of the mAbs were reactive in ELISA (Table 2). Ten of the twelve mAbs bound to the surface of bacteria as evidenced by flow cytometry. All of the mAbs were positive by Western Blot analysis with the wild type protein.

TABLE 2

| mAbs/cell lines Fusion #1 | mAbs/cell lines Fusion #2 |
|---|---|
| 1) 2H2.B8 IgG1 | |
| 2) 8H6.E11.H3 IgG2a* | |
| 3) 7H2.C11 IgG1* | |
| | 4) 2E12.A8 IgG1 |
| | 5) 8A8.B4 IgG1 |
| | 6) 3G11.D5 IgG1 |
| | 7) 13G11.C11 IgG1 |
| | 8) 13C7.D12 IgG1 |
| | 9) 1G3.B3 IgG1 |
| | 10) 9H3.E4 IgG1 |
| | 11) 3B7.G8 IgG1 |
| | 12) 3G12.A4 IgG1 |

*Not reactive in flow cytometry.
Fusion #1 was generated from E.coli produced ORF0657n-C/e antigen.
Fusion #2 was generated with Yeast expressed ORF0657n-H/y antigen.

### Example 2:

### Class Switching mAbs

All of the mAbs isolated that bound to the native antigen were of the IgG1 isotype. These antibodies were class switched to an IgG2b isotype by selecting for shift variants (Spira et al, J. of Immunogical Methods, 74:307-315, 1985). A suitable immunoassay was developed using an IgG2b conjugate and the cell line was plated at a high density. Somatic cell mutations were selected, enriched and then cloned. The binding site of the switched mAb remained identical to the original mAb, but switching to an IgG2b subtype gave a more favorable isotype (initiating the complement cascade) in the passive protection studies.

TABLE 3
Class Switched mAbs

| IgG1 isotype | IgG2b isotype |
|---|---|
| 2H2.B8 | 2H2.BE11 |
| 2E12.A8 | 2E12.BG1 |
| 8A8.B4 | 8A8.BF9 |
| 3G11.D5 | 3G11.BE5 |
| 13G11.C11 | 13G11.BF3 |
| 13C7.D12 | 13C7.BC1 |
| 1G3.B3 | 1G3.BD4 |
| 9H3.E4 | 9H3.BE4 |

### Example 3:

### Binding Inhibition Studies with Native Antigen

Purified antibodies were labeled with Alexafluor-488 using a mAb labeling kit (Molecular Probes) according to the manufacturer's instructions. The amount of mAb that would just saturate the surface of RPMI-grown bacterial cells was determined for both the labeled and unlabeled mAbs. Each of the mAbs in Table 3 (1^{st} column) were used labeled and unlabeled.

The inhibition assay was performed by first incubating 5×10⁷ cells with the unlabeled mAb at a concentration that would saturate the surface of the cells. This reaction was incubated at room temperature for 1 hour. After this incubation, the reactions were washed with 1 ml of PAA and spun at 6,000 RPM for 5 minutes in a microcentrifuge (Hermle). The supernatant was removed down to ˜50 ul and the cells were resuspended in 100 ul of PAAG containing the amount of directly labeled mAb that would just saturate the surface of the cells. After this incubation, the reactions were washed with 1 ml of PAA and spun at 6,000 RPM for 5 minutes in a microcentrifuge (Hermle). The supernatant was removed down to ˜50 ul and the cells were resuspended in 1 ml of PBS and transferred to 12×75 mm tubes for FAC analysis. As controls, separate reactions with the unlabeled mAb were measured with a secondary Alexafluor-488 conjugated goat anti-mouse IgG (H+L) (Molecular probes, 1:400 in PAAG) to determine that this mAb was bound to the surface. A positive control was also performed that had only the labeled mAb with the cells. If the unlabeled mAb bound to the same epitope as the labeled mAb then there would be no or low fluorescent reactivity associated with the cells. If the unlabeled mAb bound to a different epitope than the labeled mAb then the level of reactivity associated with the surface would be equivalent to the labeled mAb only control cells.

The panel of monoclonal antibodies fell into four reactive groups by inhibition studies:

TABLE 4

| Group I | Group II | Group III | Group IV |
|---|---|---|---|
| 2H2.B8 | 9H3.E4 | 13G11.C11 | 2E12.A8 |
| 8A8.B4 | 1G3.B3 | | 13C7.D12 |
| | 3G11.D5 | | |

### Example 4:

### Binding Studies with Denatured Antigen and Altered Antigens

ORF0657n altered proteins were used to further characterize binding. Nucleic acid encoding ORF0657n was initially cloned into the expression vector pET-28a (Novagen) and expressed in E. coli with a C-terminal 6× his tag (SEQ ID NO: 2). The expression vector with the cloned gene was subjected to mutagenesis using Stratagene's QuikChange XL Site-Directed Mutagenesis Kit following the manufacturer's instructions. The gene was mutated with specific sequential amino acid changes. The resulting plasmid was transformed into Stratagene's XL10-Gold competent cells following the manufacturer's protocol. Plasmids were isolated from transformants using Qiagen's QlAprep Spin Miniprep Kit. Transformants were screened by sequencing using ABI's 310 DNA Sequencer. Plasmid from the transformant exhibiting the greatest number of base changes was transformed into the expression host HMS174(DE3) (Novagen). Transformants were expressed following Novagen's instructions.

Different ORF0657n altered proteins were used to determine the diversity of the ORF0657n mAbs (SEQ IDs 4-19). These proteins were screened with the 10 different mAbs in dot blots using standard procedures. Positive/negatives were confirmed by Western blots using standard procedures. By this approach antibodies were grouped according to their binding profile. Seven of the antibodies resolved to three groups; the three remaining antibodies (2H2.B8, 8A8.E11.H3 and 13G11.C11) had profiles that were similar but not identical to each other (Table 5).

TABLE 5
Binding of ORFO657n specific mAbs to ORFO6S7n mutant proteins detected by Western blot

| SEQ | Group III | | Group II | | | Group IV | | Group I | | |
|---|---|---|---|---|---|---|---|---|---|---|
| ID NO: | 3G11.C11 | 3G12.A4 | 3B7.G8 | 1G3.B3 | 9H3.E4 | 2E12.A8 | 13C7.D12 | 2H2.B8 | 8A8.E11.H3 | 13G11.C11 |
| 1 | + | + | + | + | + | + | + | + | + | + |
| 2 | + | + | + | + | + | + | + | + | + | + |
| 3 | + | + | + | + | + | + | + | + | + | + |

TABLE 5
Binding of ORF0657n specific mAbs to ORF0657n mutant proteins detected by Western blot

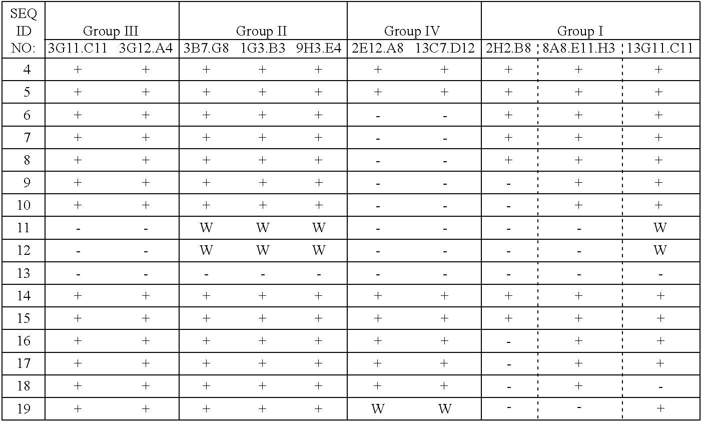

+, Antibody bound to protein in a Western;
−, Antibody did not bind to protein by Western;
W, Weak binding of antibody to protein detected by Western.
Antibodies were grouped according to hybridization profile.
A dotted line is used where similar, but not identical profiles were obtained.

### Example 5:

### BAkore Studies

In BlAcore studies the mAbs were examined by “footprint analysis” using purified ORF0657n-H/y as the antigen. Pair-wise binding experiments were conducted using real-time biomolecular interaction analysis via BIACORE®. BIACORE® incorporates microfluidics technology and surface plasmon resonance (SPR) to detect changes in mass by monitoring changes in the refractive index of a polarized light aimed directly at the surface of a carboxyl methyl dextran coated (CM5) sensor chip. The changes in response, measured in Response Units, can be correlated to the amount of bound analyte (i.e. antigen or antibody).

An anti-staphylococcal antibody (mAb 13C7.D12) was covalently bound (immobilized) on the surface of the CM5 sensor chip. The immobilized Ab was exposed first to the ORF0657n protein and subsequently to a pair of antibodies in a matrix format. After each cycle of ORF0657n protein+antibody pair, the surface of the sensor chip was regenerated back to the immobilized mAb 13C7.D12 using 20 mM HCl. Eight antibodies were tested against the ORF0657n protein in a matrix format so that all combinations of each antibody pair could be analyzed. The matrix design for mAb pairs used in this experiment is summarized in Table 6.

TABLE 6
Summary of Antibodies Tested in 8 × 8 Matrix

| | | Second Antibody | | | |
|---|---|---|---|---|---|
| Cycle # | First Antibody | Flow Cell 1 | Flow Cell 2 | Flow Cell 3 | Flow Cell 4 |
| 1 | N/A Immobilization | 13C7.D12 | 13C7.D12 | 13C7.D12 | 13C7.D12 |
| 2 | 2H2.B8 | 2H2.B8 | 13C7.D12 | 8A8.B4 | 9H3.E4 |
| 3 | 2H2.B8 | 13G11.C11 | 2E12.A8 | 1G3.B3 | 3G11.D5 |
| 4 | 13C7.D12 | 211.1382 | 13C7.D12 | 8A8.B4 | 9H3.E4 |
| 5 | 13C7.D12 | 13G11.CI1 | 2E12.A8 | 1G3.B3 | 3G11.D5 |
| 6 | 8A8.B4 | 2H2.B8 | 13C7.D12 | 8A8.B4 | 9H3.E4 |
| 7 | 8A8.B4 | 13G11.C1l | 2E12.A8 | 1G3.B3 | 3G11.D5 |
| 8 | 9H3.E4 | 2112.138 | 13C7.D12 | 8A8.B4 | 9H3.E4 |
| 9 | 9H3.E4 | 13G11.C11 | 2E12.A8 | 1G3.B3 | 3G11.D5 |
| 10 | 13G11.C11 | 2112.138 | 13C7.D12 | 8A8.B4 | 9H3.E4 |
| 11 | 13G11.C11 | 13G11.C11 | 2E12.A8 | 1G3.B3 | 3G11.D5 |
| 12 | 2E12.A8 | 2H2.B8 | 13C7.D12 | 8A8.B4 | 9H3.E4 |
| 13 | 2E12.A8 | 13G11.C11 | 2E12.A8 | 1G3B3 | 3G11.D5 |
| 14 | 1G3.B3 | 2H2.B8 | 13C7.D12 | 8A8.B4 | 9H3.E4 |
| 15 | 1G3.B3 | 13G11.C11 | 2E12.A8 | 1G3.B3 | 3G11.D5 |
| 16 | 3G11.DS | 2H2.B8 | 13C7.D12 | 8A8.B4 | 9H3.E4 |
| 17 | 3G11.D5 | 13G11.C11 | 2E12.A8 | 1G3.B3 | 3G11.D5 |

To normalize for the amount of antigen initially bound (captured) in each run, the following ratio for each test antibody/antigen complex is calculated:

$$= \frac{Test Antibody Response Units * 1000}{ORF 0657 n protein Response Units} or \frac{{mRU}_{Ab}}{{RU}_{Ag}}$$

The percentage of available epitope remaining for each antibody can be calculated for the mapping pair as follows:

$$= \frac{( {mRU}_{Ab} ( when {2}^{nd} Ab ) / {RU}_{Ag} ) * 100}{( {mRU}_{Ab} ( when {1}^{st} Ab ) / {RU}_{Ag} )} or \begin{matrix}\% Remaining \\ ( calculated for each Ab )\end{matrix}$$

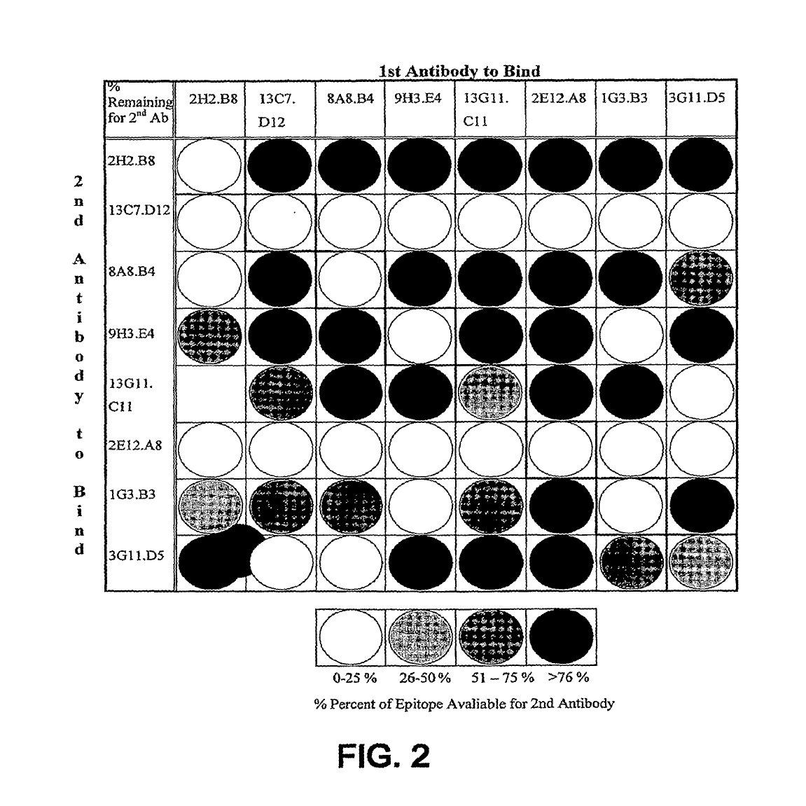

FIG. 2 illustrates matrix resulting outlining the reactivities of the monoclonal antibodies in a pair-wise binding study. The panel of monoclonal antibodies fell into three reactive areas by the BIACORE® method (See Table 7).

TABLE 7

| Group I | Group II | Group III |
|---|---|---|
| 2H2.B8 | 13G11.C11 | 13C7.D12 |
| 8A8.B4 | 3G11.D5 | 2E12.A8 |
| 9H3.E4 | | |
| 1G3.B3 | | |

### Example 6:

### Protection Studies with Passive Immunization in a Murine Sepsis Model

The monoclonal antibodies mAb 2H2.BE1 1 and mAb 13C7.BC1 were tested for their ability to provide protection against S. aureus infection. These antibodies recognize different epitopes on the ORF0657n protein. Controls included an isotype matched mAb and PBS-only.

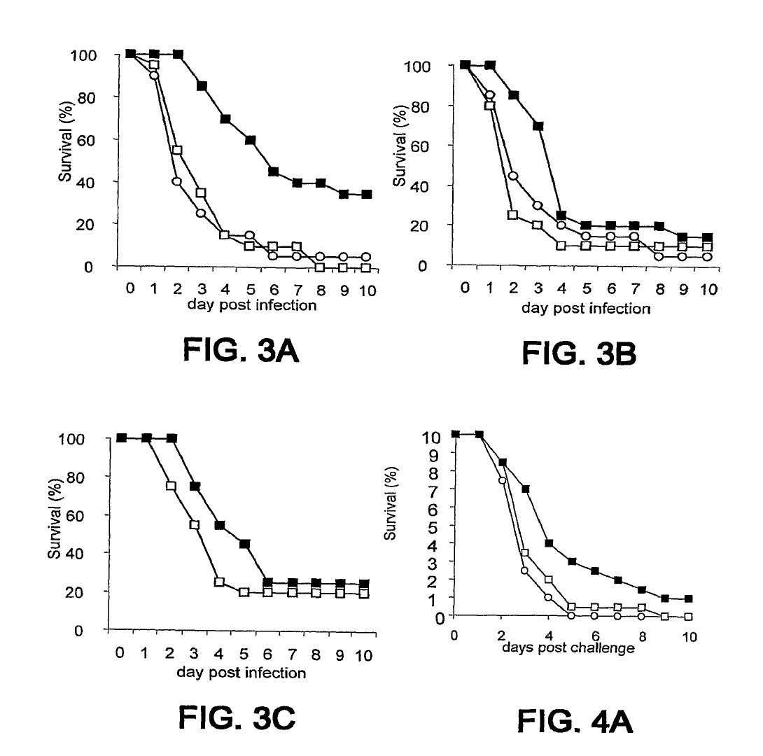

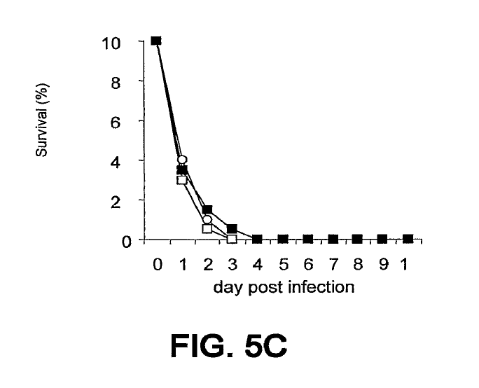

The mAbs or PBS were administered intraperitoneally (i.p.) 20 hours prior to bacterial challenge. Mice were then challenged with a LD₈₀₋₉₀ dose of S. aureus Becker i.v. and monitored for survival. Each experiment was repeated three times with groups of 10 or 20 mice and was monitored for 10 days. The half life for the monoclonal antibodies in uninfected BALB/c mice is approximately eight days. A dose of 0.5 mg was found to be optimal. The results of experiments with the two monoclonal antibodies are presented in FIGS. 3A-C, 4A, 4B, and 5A-C.

Whereas the mAb 13C7.BC1 significantly improved survival at day 10 compared to the controls in one experiment, in the other 2 repetitions the overall survival rate was similar to that of the controls (FIGS. 3A-3C). However, compared to controls, there was delay in the time to death of the mAb 13C7.BC1 treated mice within this 10 day period. A similar trend in delay of time to death of the mAb 2H2.BE1 treated mice was also noted in two of the three experiments (FIGS. 5A-5C).

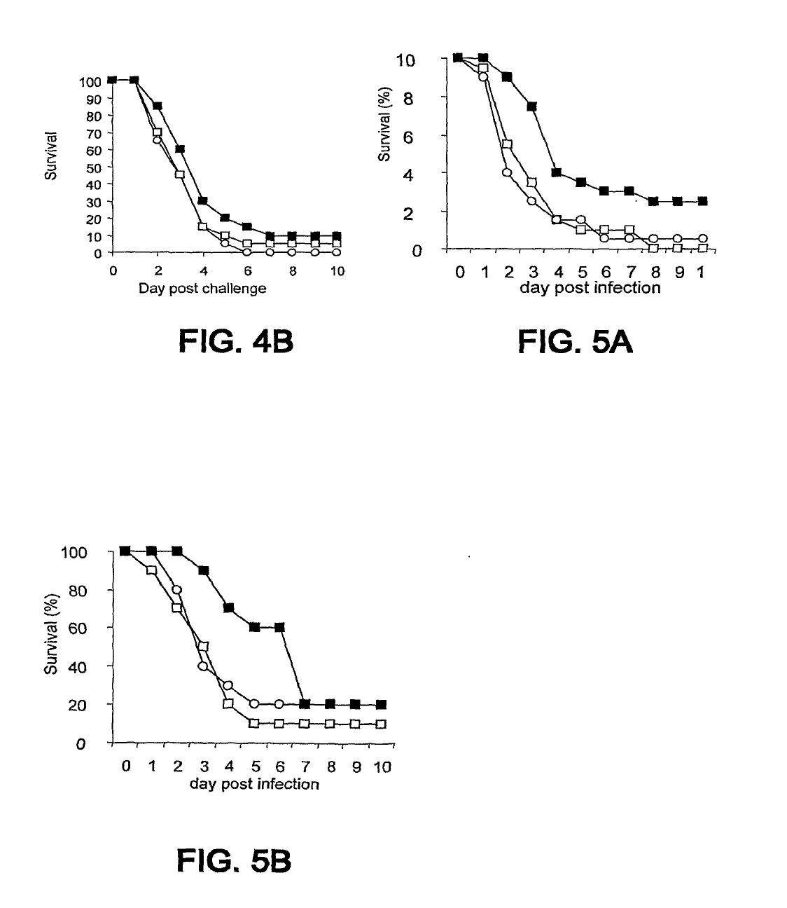

The effect of mAb 13C7.BC1 was also examined using a recent S. aureus clinical isolate UK58 (FIGS. 4A and 4B). This strain was minimally passaged from an abscess site in a patient. In two independent experiments, the results show a delay in time to death with the UK58 challenge.

Antibody persistence studies cannot be evaluated in the LD₈₀₋₉₀ model due to the rapid rate of death. Therefore, a sub-lethal challenge model was run. In the sub-lethal model the challenge dose used is 10% of that used for the LD₈₀₋₉₀ model. The sub-lethal challenge model was monitored over a four day period. Groups of 22 mice received 0.5 mg doses of either mAb 13C7.BC1 or isotype control mAb (6G6) 20 hours prior to i.v. bacterial challenge with 5×10⁷ CFU of S. aureus Becker. Two animals from each group were sacrificed just prior to challenge (T=0) to determine the mAb levels in the serum at the time of challenge. At 2, 24, 48, 72 and 96 hours post challenge, four mice from each group were sacrificed and serum mAb levels determined.

From this sub-lethal challenge experiment, the half life of mAb 13C7.BC1 in S. aureus-infected mice was estimated to be approximately one-day. In contrast, the half life of the isotype control mAb was estimated to be greater than four days (data not shown). These data point to a specific reduction of mAb 13C7.BC1 in S. aureus challenged mice, which appears to be exhausted well before the ten day period monitored in the lethal model.

In six of the eight experiments illustrated in FIGS. 3A-C, 4A, 4B, and 5A-C, improved survival was observed through approximately three days for the groups receiving the mAb administration. These results provide an indication that such mAbs have a positive effect on the survival rate of S. aureus challenged mice.

### Example 7:

### Protection Studies with Passive Immunization in a Murine Indwelling Catheter Model

A murine indwelling catheter model was used with mAb 2H2.BE11. The S. aureus strain used in this model was the clinical isolate MCL8538. This strain was selected as lower inocula could be administered while still getting reproducible colonization of catheters compared to S. aureus Becker, the strain used in the murine sepsis model.

ICR mice had catheters (PESO silicone rubber) surgically implanted into the jugular vein, held in place with sutures, and exiting with a port on the dorsal midline of the mouse. Mice were rested 9-11 days post surgery. At 24 hours prior to challenge, mice were passively immunized with a single injection of 600 mcg of murine monoclonal antibody 2H2.BE11 administered i.p. At day 0, mice were challenged with S. aureus MCL8538 administered i.v. The inoculum dose was 2-8×10⁵ CFU in 100 μl volume (Experiments 1 to 3). This low dose was found to clear spontaneously from the catheters after 4 days. Therefore, catheters were assessed for bacteria at 24 hours post challenge. At that time, mice were sacrificed and catheters harvested. The presence of bacteria on the catheters was assessed by culturing the entire catheter on TSA. If any sign of outgrowth was observed on the plate the catheter was scored as culture positive.

In two of the first three experiments, the number of culture negative catheters was significantly lower in mice passively immunized with antibody 2H2.BE11, as compared to the isotype control antibody. A fourth experiment was performed using a larger inoculum dose. In this more rigorous challenge, the dose was determined to be one in which 100% of catheters were reproducibly infected, and this infection was not spontaneously cleared by control mice (monitored over 7 days). In experiment 4, with the larger inoculum size, again, significantly fewer catheters were found to be infected in mice injected with antibody to 2H2.BE 11, compared with the isotype control. Results of the four experiments are summarized in Table 8.

TABLE 8
Number Of Culture Negative Catheters Obtained In 4 Independent Passive
Transfer Experiments Using a Murine Indwelling Catheter Model

| | Number of Culture-Negative Catheters | | | | | |
|---|---|---|---|---|---|---|
| Monoclonal | Exp#1 | Exp#2 | Exp#3 | Exp#4 | Total | p-value |
| 2H2.BE11 | 3 of 4 | 6 of 8 | 4 of10 | 4 of 9 | 17/31 | 0.0187 |
| | (75%) | (75%) | (40%) | (44%) | (54%) | |
| Isotype matched | 1 of 4 | 3 of 8 | 4 of 10 | 0 of 9 | 8/31 | |
| control | (25%) | (38%) | (40%) | (0%) | (25%) | |

Groups of ICR mice with indwelling catheters were injected i.p. with 600 mcg of murine monoclonal antibody 24 hours prior to challenge, all monoclonals of the IgG2b isotype

### Example 8:

### Ex-Vivo Pre-Opsonization of Bacteria Using anti-ORF0657n Monoclonal Antibodies

2H2.B8 (IgG1), 2H2.BE11 (IgG2b), or 13C7.IgG2b or Isotype Matched Control mAbs

To test whether monoclonal antibodies to ORF0657n are opsonic, passive protection experiments were conducted in which a lethal dose of S. aureus was pre-opsonized with the monoclonal antibodies 2H2.B8, 2H2.BE11, or 13C7.IgG2b, or an isotype matched control monoclonal antibody. Pre-opsonized bacteria were then administered to mice i.p. Bacteria used in these experiments were S. aureus RN4220 (wild type) or RN4220.0657n. The RN4220.0657n bacteria were engineered to express ORF0657n in the absence of control by the FUR box. Therefore, they could be grown in the presence of iron and still express ORF0657n antigen on their surface. Alternatively, RN4220 (wild type) was passed 2× in a low iron medium RPMI to induce expression of 0657n on the bacteria surface.

A quantity of bacteria sufficient for 6 Balb/c mice (6×LD₁₀₀) was incubated with 800 μg IgG at 4 ° C. for 1 hour, with gentle rocking. Bacteria were then pelleted and any unbound mAb removed. Antibody-opsonized bacteria were re-suspended in 2.4 mL of PBS, and 0.4 mL (1×LD₁₀₀) was injected into each of five mice. After challenge, each inoculum was quantitated by plating on TSA to insure that equivalent CFU was given to all groups of mice and that the mAbs had not aggregated the bacteria. Survival was monitored for 3 days post challenge. Since the target antigen must be present on the surface of the bacteria for this procedure to be effective, care was taken to ensure that 0657n was expressed on the bacteria prior to opsonization. ORF0657n expression was monitored by flow cytometry using mAb 2H2.B8. The dose of opsonized bacteria injected into each mouse was 2-4×10⁹ CFU RN4220.0657n/mouse, or 1-2 X 10′9 CFU RN4220(2X RPMI)/mouse.

When pre-opsonized with either 2H2.B8 or 2H2.BE1 1, but not an isotype matched control mAb, mice were protected from death from a lethal dose of RN4220.0657n staphylococci. The experiment was repeated twice for the IgG1 isotype and three times for the IgG2b isotype with similar results (Table 9A).

TABLE 9A
Ex-vivo Protection with Anti-0657n mAb

| | Exp 1 | Exp 2 | Exp 3 | |
|---|---|---|---|---|
| | Surviving | Surviving | Surviving | |
| Monoclonal | Mice | Mice | Mice | Total |
| 2H2.BE11 (IgG2b) | 5 | 4 | 5 | 93% (14/15) |
| 6G6.A8(IgG2b) | 1 | 0 | 1 | 13% (2/15) |
| PBS | 1 | 2 | 0 | 20% (3/15) |
| 2H2.BE11 (IgG1) | ND | 4 | 5 | 90% (9/10) |
| 10B4.H4 (IgG1) | ND | 1 | 1 | 20% (2/10) |

Five mice were used in each experiment. Challenge strain RN4220.0657n.pYZ1 19. Dose: 2-4 × 10⁹ CFU. Test mAbs: murine anti-0657n 2H2.BE11 (IgG2b); 2H2.B8 (IgG1).

When pre-opsonized with either mAb 2H2.B8 but not an isotype matched control mAb, mice were protected from death from a lethal dose of RN4220 (2X RPMI) staphylococci. The experiment was repeated six times with similar results (Table 9B).

TABLE 9B
Ex-vivo Protection with Anti-0657n mAb

| Monoclonal | # Tests | Aggregate | % Survival |
|---|---|---|---|
| 2H2.B8 | 6 | 30/30 | 100% |
| 10B4.IgG1 | 6 | 2/30 | 7% |
| Isotype control | | | |
| 13C7.IgG2b | 2 | 0/10 | 0% |
| 6G6.IgG2b | 2 | 0/10 | 0% |
| Isotype control | | | |

Murine anti-0657n 2H2 was very effective in preventing death in this lethal model. The 13C7 mAb was not effective in this model (as opposed to the previously described model illustrated in FIGS. 3-6). All (2H2.BE11, 2H2.B8 and 13C7.IgG2b) of the anti-0657n mAb's bind RN4220 (as demonstrated using flow cytometery) and all have opsonizing activity in the in vitro OPA assay. This model reflects an additional requirement for epitope specificity for enhancing survival in the peritoneum of the mouse.

### Example 8:

### Epitope Mapping Studies Performed with 2H2 mAb

The experiments described in this example provide evidence that the monoclonal antibody 2H2.BE11 recognizes a conformational epitope within ORFO657n. The experiments localized the minimal sequence within ORFO657n required for displaying the conformational epitope in a three dimensional structure recognized by 2H2 mAb. In addition, the experiments identified distinct lysine residues within the minimal sequence of ORFO657n that become protected from reacting with small molecules when 2H2 mAb is bound to ORFO657n.

The potential ability of 2H2 mAb to recognize linear epitopes of typically 9 to 14 amino acids in length within the sequence of ORFO657n was investigated using epitope extraction and starting with an ORF0657n fragment from amino acid 42 to amino acid 486 of SEQ ID NO: 1 (“ORF0657t”). In detail: 30 ug of 2H2 mAb were immobilized by chemical cross linking to 10 mg of cyanogen bromide activated sepharose (Amersham cat. No. 17 0430 01) for each of the epitope extraction experiments. Proteolytic digests of the ORF0657t were generated with GIuC (Roche Applied Science cat. No. 11 420 3997 001), Asp-N (Roche Applied Science cat. No. 11 054 589 001) or Chymotrypsin (Roche Applied Science cat. No. 11 418 467 001) and characterized by 1D/LC-MS/MS on a linear ion trap (LTQ—Thermo Electron Inc). In three individual experiments 8.4 μg of the characterized proteolytic digest from any protease was allowed to react with the immobilized antibody. Unbound peptides were washed off the antibody cross-linked beads. Potentially bound peptides were eluted with low pH and characterized by ID/LC-MS/MS. None of the generated proteolytic peptides were recognized with high efficiency and specificity by 2H2 mAb, providing a strong indication that 2H2 mAb did not recognize a linear epitope.

The finding that 2H2 mAb did not recognize a linear sequence of ORF0657n was corroborated by a limited chemical cleavage experiment. ORF0657t was chemically cleft with CNBr for 2 hours. The resulting cleavage products were analyzed by SDS-PAGE. SDS-PAGE analysis showed 5 major bands with molecular weights of approximately 42 kDa, 35 kDa, 25 kDa, 15 kDa and 10 kDa. A Western Blot analysis with 2H2 mAb clearly showed that only the 42 kDa band was recognized by 2H2. All bands were excised from the SDS-PAGE, in-gel digest was performed, and the resulting peptides that were identified by tandem mass spectrometry were matched to corresponding sequences in ORF0657t. The result of the analysis of the major bands is shown in Table 10:

TABLE 10

| | Binds to | | Calculated |
|---|---|---|---|
| CNBr cleavage | 2H2 mAb | ORFO657t | MW kDa |
| Band 42 kDa | yes | [001-356] | 40.7 |
| Band 35 kDa | no | [001-323] | 36.7 |
| Band 25 kDa | no | [001-214] | 23.9 |
| | | [116-302] | 21.9 |
| Band 15 kDa | no | [215-356] | 16.8 |
| | | [303-446] | 16.6 |
| Band 10 kDa | no | [114-214] | 11.7 |
| | | [215-302] | 10.39 |
| | | [357-446] | 10.28 |

The importance of a fragment with a molecular weight of 42 kDa was confirmed by epitope excision. In detail, 210 μg of 2H2 mAb was immobilized by chemical cross linking to 50 mg of cyanogen bromide activated sepharose (Amersham cat. No. 17 0430 01) for each of the epitope excision experiments. Then, 50 μg of intact ORF0657t was allowed to bind to the immobilized antibody and non-bound ORF0657t washed off by intensive washing with phosphate buffered saline. In three independent experiments proteases Glu-C, Trypsin and a sequential combination of GIuC, AspN, Trypsin, Chymotrypsin, and Carboxy-peptidase Y were added for 5 hours or one hour per protease in the sequential combination. Peptides that were excised by the proteases during the incubation were thoroughly washed away and ORF0657t fragments that specifically bound to 2H2 mAb released with SDS loading buffer.

Fragments that specifically bound to 2H2 mAb were analyzed by SDS-page. All three of the epitope excision experiments showed exclusively one band with a molecular weight between 40 and 42 kDa in the SDS-Page analysis. Bands binding to 2H2 mAb were confirmed by Western Blot analysis. The epitope excision experiment was repeated for the Glu-C protease. This time the fragment of ORF0657t that specifically bound to 2H2 mAb was released with acidic conditions and analyzed by 1D/LC-MS/MS on a linear ion trap (LTQ, Thermo Electron). The eluted sample showed a signal (total ion count) with the expected intensity at 82-87 minutes (40%-45% acetonitrile) and multiple charge states ([M+67 H]⁶⁷⁺ to ([M+30 H]³⁰⁺) that deconvoluted to 42.628 kDa. A possible fragment of ORFO657t corresponding to this particular mass is sequence [012-382] of ORFO657t with a molecular weight of 42.6 kDa.

To determine which lysine residues of ORF0657t are protected from chemical reactions upon binding of 2H2 mAb, chemical labeling experiments were preformed with sulfo-NHS-acetate (Pierce Cat. No. 26777) using three different experimental conditions in the presence or absence of 2H2 mAb. See Table 11.

TABLE 11

| | Experiment | | |
|---|---|---|---|
| | 1 | 2 | 3 |
| molar excess | 0 or 3 | 0 or 3 | 0 or 3 |
| 2H2 mAb | | | |
| molar excess | 25 | 500 | 75 |
| sulfo-NHS acetate | | | |
| Reaction temper- | room | 15 | 37 |
| ature ° C. | temperature | | |
| Reaction time | 1 hour | 30 minutes | 2 hours |

For each experiment, reaction products produced with 0 or 3 molar excess 2H2 mAb were incubated with one of three proteases resulting in 2×9 reaction mixtures. Experiment 1 employed GluC, AspN and Trypsin. Experiments 2 and 3 employed GIuC, AspN, and Chymotrypsin. The proteolytic peptides were then analyzed by 1D/LC-MS/MS. For each of the reactions a ratio of acetylated and non-acetylated lysine residues was calculated based on the area under curve of the total ion count (TIC) of the individual peptides. Obtained ratios were then compared between the pairs (with and without 2H2 mAb) for identical reaction conditions. A global analysis was performed for all three reaction conditions to identify lysine residues within ORF0657t that are maximally shielded upon binding of ORF0657t to 2H2 mAb. The chemical labeling experiment described above identified K76, K257 and potentially K443 as being most protected upon binding of 2H2 mAb. Protection against chemical labeling is likely due to direct binding. However, it is possible that such protection could be due to binding in close proximity to the protected sites or by long range structural changes within

ORF0657nI

In summary, the above described experiments provide clear evidence that the epitope within ORF0657t that is recognized by the 2H2 mAb is conformational. The fragment of ORF0657t that is recognized by 2H2 mAb has an N-terminus located between amino acids 1 and 115 of ORF0675t and a carboxyl terminus located between amino acids 323-357 of ORF0657t. Even though it can not be excluded that protection from chemical labeling upon binding of 2H2 mAb is influenced by long range structural changes, it is very likely that areas in close proximity to Lysine 76 and Lysine 275 participate in direct antibody interaction.

### Example 9:

### 2H2 mAb Sequence Identification

Identification of the variable light (V₁) and variable heavy (Vₕ) sequences of hybridoma expressed 2H2 IgG was accomplished by combining the degenerative primer PCR/overlap extension cloning process for single chain variable fragments (scFv) assembly (Krebber et al. JIM 201(1):35-55, 1997), with high throughput screening of soluble scFv fused to a human kappa light chain constant domain or scAb material via Biacore. This allowed for fine discrimination of mutations in V₁ frameworks 1, 4 and Vₕ frameworks 1, 4 generated by the degenerative primer method.

Briefly, RNA material was purified from the hybridoma cell line using standard methods from a Total RNA Kit™ (Ambion Inc.). This material was then reverse transcribed to cDNA and utilized as template in PCR to amplify the variable regions. The conditions for the PCR amplification of the V₁ and Vₕ chains was based upon the protocol described by Krebber et aL JIM 201(1):35-55, 1997. The primers are designed such that a (Gly4Ser)₄ linker (SEQ ID NO: 32) is added which provides domains for a third PCR reaction in which the Vₕ and V₁ are overlapped to create a V₁ (Gly4Ser)₄-Vₕ scFv.

The first set of PCR reactions to amplify the variable chains individually, were carried out in a volume of 100 μl containing 5 μl of the cDNA reaction, 2 μM each of the forward and reverse primer sets for amplification of V₁ and Vₕ, and a high fidelity PCR master mix. The reactions were denatured for 4 minutes at 94° C. followed by 30 cycles of 30 seconds at 94° C., 30 seconds at 50° C., 1 minute at 72° C., and finished at a final cycle of 5 minutes at 72° C. The full length PCR products were gel purified.

To construct the full length product a third PCR reaction was done to assemble to scFv from the amplified Vₕ and V₁ material. In a volume of 100 p.1 approximately 20 ng each of Vₕ and V₁ DNA and a high fidelity PCR master mix was denatured for 5 minutes at 94° C., followed by 3 cycles of 30 seconds at 94° C., 30 s at 60° C., and 30 seconds at 72° C. in the absence of primers. The modified PCR primers, SEQ ID NO: 33 and SEQ ID NO: 34 were added at a final concentration of 1 μM, and 30 cycles of 30 seconds at 94° C., 1 minutes at 60° C., and 1 minute at 72° C. were performed, followed by 7 minutes at 72° C. The expected full length scFv PCR products were gel purified.

The amplified scFv material was cloned into the MP16 soluble expression vector for scAb production (Hayhurst et al., JIM 276(1-2):185-196, 2003) and sequence analysis. A single restriction enzyme digest with Sfil was used for directional cloning into the MP16 vector. Clones with apparent full length variable heavy and variable light chains present were then expressed as scAbs in XL1-Blue cells and recovered from the periplasm using a standard osmotic shock procedure. Briefly, clones were grown at 37° C. overnight in growth media containing 2% glucose and 100 μg/ml ampicillin in a 96 well format. 20 μl of the overnight culture was transferred to new media containing 0.1% glucose and 100 μg/ml ampicillin and grown until an OD₆₀₀ of 0.6 was reached. The cells were induced for scAb expression by adding IPTG at a final concentration of 0.5 mM and incubated overnight while shaking at 150 rpm, at room temperature. The scAbs were purified from the cells using a Qiagen Ni-NTA superflow robotic procedure.

To analyze each scAb periplasmic preparation for binding activity to ORF0657t, a Biacore3000 surface plasmon resonance (SPR) instrument (Upsala, Sweden) was utilized. Standard EDC/NHS coupling was used to covalently mobilize approximately 250 resonance units of the 0657t antigen directly to the experimental flow cell surface of a CM5 sensor chip. A reference flow cell surface was activated and deactivated without coupling of protein. Each preparation was then run over the surface and association and dissociation of the scAb to antigen was measured. The surfaces were regenerated between runs by a single injection of 10 mM HCl for 20 seconds at a flow rate of 20 μl/min, followed by a 2 minute stabilization period. All samples were run in duplicate and buffer only runs were used as controls. After screening 95 clones, a clone was selected based on its binding activity. The final 2H2 clone chosen was based upon its similar affinity for ORF0657t as the original hybridoma prepared IgG material as well as comparative sequence analysis.

The amino sequence of the 2H2 Vₕ (SEQ ID NO: 20) and V₁ (SEQ ID NO: 21) were as follows:

| 2H2 Vₕ Amino Acid Sequence | |
|---|---|
| (SEQ ID NO: 20) | |
| 1 | DVHLVESGPG LVAPSQNLSI TCTVSGFSLS RYGVHWVRQP PGKGLEWLGL |
| 51 | IWAGGVTIYN STLMSRLSIS KDSSKSQVFL KMNSLQIDDT AIYYCAREAS |
| 101 | RDBYFDYWGQ GTTLTVSS |
| 2H2 Vₗ Amino Acid Sequence | |
| (SEQ ID NO: 21) | |
| 1 | DIVMTQSPAI MSASPGEKIT MTCSASSSVS YIYWYQQKSG TSPKRWIYDT |
| 51 | SKLASGVPFR FSGGGSGTSF SLTISSMEAE DAATYYCQQW SSNPLTFGAG |
| 101 | TKLEIK |

The underlined portions are the CDR's. CDR's were identified based on the Kabat definition. The encoding nucleic acid sequence is provided by SEQ ID NO: 24 (Vₕ) and SEQ ID NO: 25 (Vi).

### Example 10:

### 2H2 IgG Chimera Expression

The variable regions for 2H2 mAb were cloned from mouse hybridoma as described in Example 9. The sequences for the variable regions were PCR amplified and DNA encoding the heavy chain variable regions were fused in-frame with DNA encoding the IgG1 constant region whereas DNA encoding the light chain variable region were fused in-frame with DNA encoding the kappa constant region. The cloning procedure for the resulting antibody expression vectors is described below.

The variable regions were PCR amplified. PCR reactions were carried out in a volume of 25 μl containing high fidelity PCR master mix, template volume 1 μl and forward and reverse primers: 1 μl each. PCR condition was 1 cycle of 94° C., 2 minutes, 25 cycles of 94° C., 1.5 minutes; 60° C., 1.5 minutes; 72° C., 1.5 minutes and 72° C., 7 minutes; 4° C. until removed and cloned in-frame with leader sequence at the 5′-end and constant region at the 3′-end using In-Fusion strategy. The following primers were used: Light chain forward, 5′-ACAGATGCCAGATGCGATATTGTGATGACCCAGTCT (SEQ ID NO: 28); Light chain reverse, 5′-TGCAGCCACCGTACGTTTTATTTCCAGCTTGGTCCC (SEQ ID NO: 29); Heavy chain forward, 5′-ACAGGTGTCCACTCGGATGTGCACCTGGTGGAGTCA (SEQ ID NO: 30); and Heavy chain reverse, 5′-GCCCTTGGTGGATGCCGAGGAGACTGTGAGAGTGGT (SEQ ID NO: 31). The DNA sequences for all the clones were confirmed by sequencing.

The amino acid sequences deduced from DNA sequences are:

| Mouse 2H2 Variable and Human Kappa Constant Region Amino Acid Sequence | |
|---|---|
| (SEQ ID NO: 22) | |
| 1 | DIVMTQSPAI MSASPGEKIT MTCSASSSVS YIYWYQQKSG TSPKRWIYDT |
| 51 | SKLASGVPFR FSGGGSGTSF SLTISSMEAE DAATYYCQQW SSNPLTFGAG |
| 101 | TKLEIKRTVA APSVFIFPPS DEQLKSGTAS VVCLLNNFYP REAKVQWKVD |
| 151 | NALQSGNSQE SVTEQDSKDS TYSLSSTLTL SKADYEKHKV YACEVTHQGL |
| 201 | SSPVTKSFNR GEC |
| Mouse 2H2 Variable and Human IgGl Constant Region Amino Acid Sequence | |
| (SEQ ID NO: 23) | |
| 1 | DVHLVESGPG LVAPSQNLSI TCTVSGFSLS RYGVHWVRQP PGKGLEWLGL |
| 51 | IWAGGVTIYN STLMSRLSIS KDSSKSQVFL KMNSLQIDDT AIYYCAREAS |
| 101 | RDHYFDYWGQ GTTLTVSSAS TKGPSVFPLA PSSKSTSGGT AALGCLVKDY |
| 151 | FPEPVTVSWN SGALTSGVHT FPAVLQSSGL YSLSSVVTVP SSSLGTQTYI |
| 201 | CNVNHKPSNT KVDKRVEPKS CDKTHTCPPC PAPELLGGPS VFLFPPKPKD |
| 251 | TLMTSRTPEV TCVVVDVSHE DPEVKFNWYV DGVEVHNAKT KPREEQYNST |
| 301 | YRVVSVLTVL HQDWLNGKEY KCKVSNKALP APIEKTISKA KGQPREPQVY |
| 351 | TLPPSREEMT KNQVSLTCLV KGFYPSDIAV EWESNGQPEN NYKTTPPVLD |
| 401 | SDGSFFLYSK LTVDKSRWQQ GNVFSCSVMH EALHNHYTQK SLSLSPGK |

The variable regions are underlined.

The antibodies were expressed in 293EBNA monolayer cells. The plasmids were transfected using PEI based transfection reagents. The transfected cells were incubated in Opti-MEM serum free medium and the secreted antibodies were purified from medium using protein A/G affinity chromatography. The concentration of purified antibodies was determined by OD280 nm and the purity was measured by LabChip™ capillary electrophoresis.

The expression of both light and heavy chains was driven by human CMV promoter and bovine growth hormone polyadenylation signal. (Shiver et al., Ann. N.Y. Acad. Sci., 772:198-208, 1995.) The leader sequence in the front mediated the secretion of antibodies into the culture medium. The leader sequence for the heavy chain was MEWSWVFLFFLSVTTGVHS (SEQ ID NO: 26) and for the light chain was MSVPTQVLGLLLLWLTDARC (SEQ ID NO: 27). The expression vectors carry oriP from EBV viral genome for prolonged expression in 293EBNA cells and the bacterial sequences for kanamycin selection marker and replication origin in E. coli.

The antibodies were expressed in 293EBNA monolayer cells. The plasmids were transfected using PEI based transfection reagents. The transfected cells were incubated in Opti-MEM serum free medium and the secreted antibodies were purified from medium using protein A/G affinity chromatography. The concentration of purified antibodies was determined by OD280nm and the purity by LabChip capillary electrophoresis.

### Example 11:

### Affinity Determination

Comparative analysis was performed on 2H2 mAb as hybridoma material, scAb and a chimeric antibody. 2H2 mAb Vₕ and V₁ region were cloned and expressed as an IgG chimera as described in Example 10. scAb was cloned into the MP16 vector (Example 9), which produces a scFv with a Human Kappa chain tag fused to it. As further described below, the antigen affinity was not significantly different among the constructs.

To measure a 1:1 interaction between the binding domain and the antigen, the experimental set up on Biacore was modified depending on whether antibody fragment or full length IgG was analyzed. For IgG measurements, the IgG was captured to the surface as ligand and ORF0657t was run as analyte. For antibody fragment analysis, ORF0657t was bound to the surface and the antibody fragment was run as the analyte. This demonstrated that the affinity of the original 2H2 mAb hybridoma material to the ORF0657t antigen shows no significant change upon recombinant cloning (Table 12). Data were acquired via surface plasmon resonance on a Biacore 3000; each analyte was run at multiple concentrations, with two replicates per concentration. Data were analyzed with BIAevaluation (Biacore, Inc.) with simultaneous fits of entire concentration series. Fit parameters are listed in Table 12.

TABLE 12

| | On-rate ka | Off-rate | Affinity, | chi² |
|---|---|---|---|---|
| | (1/Ms) | kd (1/s) | KD | global fit |
| 2H2 murine IgG2b | 6.10E+04 | 2.01E−03 | 33 nM | 0.902 |
| 2H2 scAb | 4.91E+04 | 1.91E−03 | 39 nM | 0.429 |
| 2H2 IgG chimera | 1.10E+05 | 2.73E−03 | 25 nM | 0.295 |

### Example 12:

### ORF0657n Based Sequences

The highlighted amino acids (indicated by bold and underlying) present in SEQ ID NOs: 4-19 show amino acid alterations to ORF0657n:

0657n
(SEQ ID NO: 1)
MNKQQKEFKSFYSIRKSSLGVASVATSTLLLLMSNGEAQAAAEETGGTNTEAQPKTEAVASPTTTSEKAPETKPVAN
AVSVSNKEVEAPTSETKEAKEVKEVKAPKETKAVKPAAKATNNTYPILNQELREAIKNPAIKDKDHSAPNSRPIDFE
MKKENGEQQFYHYASSVKPARVIFTDSKPEIELGLQSGQFWRKFEVYEGDKKLPIKLVSYDTVKDYAYIRFSVSNGT
KAVKIVSSTHFNNKEEKYDYTLMEFAQPIYNSADKFKTEEDYKAEKLLAPYKKAKTLERQVYELNKIQDKLPEKLKA
EYKKKLEDTKKALDEQVKSAITEFQNVQPTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
0657nC/e
(SEQ ID NO: 2)
MNKQQKEFKSFYSIRKSSLGVASVAISTLLLLMSNGEAQAAAEETGGTNTEAQPKTEAVASPTTTSEKAPETKPVAN
AVSVSNKEVEAPTSETKEAKEVKEVKAPKETKAVKPAAKATNNTYPILNQELREAIKNPAIKDKDHSAPNSRPIDFE
MKKENGEQQFYHYASSVKPARVIFTDSKPEIELGLQSGQFWRKFEVYEGDKKLPIKLVSYDTVKDYAYIRFSVSNGT
KAVKIVSSTHFNNKEEKYDYTLMEFAQPIYNSADKFKTEEDYKAEKLLAPYKKAKTLERQVYELNKIQDKLPEKLKA
EYKKKLEDTKKALDEQVKSAITEFQNVQPTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMAILLALSSIVAFVLPRKRKNLEHHHHHH
0657nH/y
(SEQ ID NO: 3)
MAEETGGTNTEAQPKTEAVASPTTTSEKAPETKPVANAVSVSNKEVEAPTSETKEAKEVKEVKAPKETKEVKPAAKA
TNNTYPILNQELREAIKNPAIKDKDHSAPNSRPIDFEMKKKDGTQQFYHYASSVKPARVIFTDSKPEIELGLQSGQF
WRKFEVYEGDKKLPIKLVSYDTVKDYAYIRFSVSNGTKAVKIVSSTHFNNKEEKYDYTLMEFAQPIYNSADKFKTEE
DYKAEKLLAPYKKAKTLERQVYELNKIQDKLPEKLKAEYKKKLEDTKKALDEQVKSAITEFQNVQPTNEKMTDLQDT
KYVVYESVENNESMMDTFVKHPIKTGMLNGKKYMVMETTNDDYWKDFMVEGQRVRTISKDAKNNTRTIIFPYVEGKT
LYDAIVKVHVKTIDYDGQYHVRIVDKEAFTKANTDKSNKKEQQDNSAKKEATPATPSKPTPSPVEKESQKQDSQKDD
NKQLPSVEKENDASSESGKDKTPATKPTKGEVESSSTTPTKVVSTTQNVAKPTTASSKTTKDVVQTSAGSSEAKDSA
PLQKANIKNTNDGHTQSQNNKNTQENKAKS
SEQ ID NO: 4
MNKQQKEFKSFYSIRKSSLGVASVAISTLLLLMSNGEAQAAAEETGGTNTEAQPKTEAVASPTTTSEKAPETKPVAN
AVSVSNDEVEAPTSETKEAKEVKEVKAPKETKEVKPAAKATNNTYPILNQELREAIKNPAIKDKDHSAPNSRPIDFE
MKKKDGTQQFYHYASSVKPARVIFTDSKPEIELGLQSGQFWRKFEVYEGDKKLPIKLVSYDTVKDYAYIRFSVSNGT
KEVKIVSSTHFNNKEEKYDYTLMEFAQPIYNSADKFKTEEDYKAEKLLAPYKKAKTLERQVYELNKIQDKLPEKLKA
EYKKKLEDTKKALDEQVKSAITEFQNVQPTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 5
MNKQQKEFKSFYSIRKSSLGVASVAISTLLLLMSNGEAQAAAEETGGTNTEAQPKTEAVASPTTTSEKAPETKPVAN
AVSVSNKEVEAPTSETKEAKEVKEVKAPKETKEVKPAAKATNNTYPILNQELREAIKNPAIKDKDHSAPNWRPIDFE
MKKKDGTQQFYHYASSVEPARVIFTDSKPEIELGLQSGQFWRKFEVYEGDKKLPIKLVSYDTVKDYAYIRFSVSNGT
KEVKIVSSTHFNNKEEKYDYTLMEFAQPIYNSADKFKTEEDYKAEKLLAPYKKAKTLERQVYELNKIQDKLPEKLKA
EYKKKLEDTKKALDEQVKSAITEFQNVQPTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 6
MNKQQKEFKSFYSIRKSSLGVASVAISTLLLLMSNGEAQAAAEETGGTNTEAQPKTEAVASPTTTSEKAPETKPVAN
AVSVSNKEVEAPTSETKEAKEVKEVKAPKETKEVKPAAKATNNTYPILNQELREAIKNPAIKDKDHSAPNWRPIDFE
MKKKDGTQQFYHYASSVEPARVIFTDSKPEIELGLQSGQFWRKFEVYEGDKKLPIKLVSYDTVKDYAYIRFSVSNGT
KAVKIVSSTHFNNKEEKYDYTLMEFAQPIYNSADKFKTEEDYKAEKLLAPYKKAKTLERQVYELEKIQDKLPEKLKA
EYKKKLEDTKKALDEQVKSAITEFQNVQPTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKFTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 7
MNKQQKEFKSFYSIRKSSLGVASVAISTLLLLMSNGEAQAAAEETGGTNTEAQPKTEAVASPTTTSEKAPETKPVAN
AVSVSNKEVEAPTSETKEAKEVKEVKAPKETKEVKPAAKATNNTYPILNQELREAIKNPAIKDKDHSAPNWRPIDFE
MKKKDGTQQFYHYASSVEPARVIFTDSKPEIELGLQSGQFWRKFEVYEGDKKLPIKLVSYDTVKDYAYIRFSVSNGT
KEVKIVSSTHFNNKEEKYDYTLMVFAQPIYNSADKFKTEEDYKAEKLLAPYKKAKTLERQVYELNKIQDKLPEKLKA
EYKKKLEDTKKALAEQVKSAITEFQNVQPTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 8
MNKQQKEFKSFYSIRKSSLGVASVAISTLLLLMSNGEAQAAAEETGGTNTEAQPKTEAVASPTTTSEKAPETKPVAN
AVSVSNDEVEAPTSETKEAKEVKEVKAPKETKEVKPAAKATNNTYPILNQELREAIKNPAIKDKDHSAPNWRPIDFE
MKKKDGTQQFYHYASSVEPARVIFTDSKPEIELGLQSGQFWRKFEVYEGDKKLPIKLVSYDTVKDYAYIRFSVSNGT
KEVKIVSSTHFNNKEEKYDYTLMEFAQPIYNSADKFKTEEDYKAEKLLAPYKKAKTLERQVYELEKIQDKLPEKLKA
EYKKKLEDTKKALAEQVKSAITEFQNVQPTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 9
MNKQQKEFKSFYSIRKSSLGVASVAISTLLLLMSNGEAQAAAEETGGTNTEAQPKTEAVASPTTTSEKAPETKPVAN
AVSVSNDEVEAPTSETKEAKEVKEVKAPKETKEVKPAAKATNNTYPILNQELREAIKNPAIKDKDHSAPNWRPIDFE
MKKKDGTQQFYHYASSVEPARVIFTKSKPEIELGLQSGQFWRKFEVYEGDKKLPIKLVSYDTDKDYAYIRFSVSNGT
KEVKIVSSTHFNNKEEKYDYTLMEFAQPIYNSADKFKTEEDYKAEKLLAPYKKAKTLERQVYELNKIQDKLPEKLKA
EYKKKLEDTKKALAEQVKSAITEFQNVQPTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 10
MNKQQKEFKSFYSIRKSSLGVASVAISTLLLLMSNGEAQAAAEETGGTNTEAQPKTEAVASPTTTSEKAPETKPVAN
AVSVSNDEVEAPTSETKEAKEVKEVKAPKETKEVKPAAKATNNTYPILNQELREAIKNPAIKDKDHSAPNWRPIDFE
MKKKDGTQQFYHYASSVEPARVIFTKSKPEIELGLQSGSTWRKFEVYEGDKKLPIKLVSYDTDKDYAYIRFSVSNGT
KEVKIVSSTHFNNKEEKYDYTLMVFAQPIYNSADKFKTEEDYKAEKLLAPYKKAKTLERQVYELEKIQDKLPEKLKA
EYKKKLEDTKKALAEQVKSAITEFQNVQPTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 11
MNKQQKEFKSFYSIRKSSLGVASVAISTLLLLMSNGEAQAAAEETGGTNTEAQPKTEAVASPTTTSEKAPETKPVAN
AVSVSNDEVEAPTSETKEAKEVKEVKAPKETKEVKPAAKATNNTYPILNQELREAIKNPAIKDKDHSAPNWRPIDFE
MKNDKGTQQFYHYASSVEPARVIFTKSKPEIELGLQSGQFWRKFEVYEGDKKLPIKLVSYDTDKDYAYIRFSVSNGT
KEVKIVSSTHFNNKEEKYDYTLMVFAQPIYNSADKFKTEEDYKAEKLLAPYKKAKTLERQVYELEKIQDKLPEKLKA
EYKKKLEDTKKALAEQVKSAITEFQNVQPTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 22
MNKQQKEFKSFYSIRKSSLGVASVAISTLLLLMSNGEAQAAAEETGGTNTEAQPKTEAVASPTTTSEKAPETKPVAN
AVSVSNDEVEAPTSETKEAKEVKEVKAPKETKEVKPAAKATNNTYPILNQELREAIKNPAIKDKDHSAPNWRPIDFE
MKNDKGTQQFYHYASSVEPARVIFTKSKPIIELGLQSGQFWRKFEVYEGDKKLPIKLVSYDTDKDYAYIRFSVSNGT
KEVKIVSSTHFNNKEEKYDYTLMVFAQPIYNSADKFKTEEDYKAEKLLAPYKKAKTLERQVYELEKIQDKLPEKLKA
EYKKKLEQTKKALAEQVKSAITEFQNVQPTNEKMTDLQDAHYVVYESVENSESMMDTFVKHPIKTGMLNGKKYMVME
TTNDDYWKDFMVEGQRVRTISKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 13
MNKQQKEFKSFYSIRKSSLGVASVAISTLLLLMSNGEAQAAAEETGGTNTEAQPKTEAVASPTTTSEKAPETKPVAN
AVSVSNDEVEAPTSETKEAKEVKEVKAPKETKEVKPAAKATNNTYPILNQELREAIKNPAIKDKDHSAPNWRPIDFE
MKNDKGTQQFYHYASSVEPARVIFTKSKPIIELGLQSGQFWRKFEVYEGDKKLPIKLVSYDTDKDYAYIRFSVSNGT
KEVKIVSSTHFNNKEEKYDYTLMVFAQPIYNSADKFKTEEDYKAEKLLAPYKKAKTLERQVYELEKIQDKLPEKLKA
EYKKKLEQTKKALAEQVKSAITEFQNVQPTNEKMTDLQDAHYVVYESVENSESMMDTFVKHPIKTGMLNGKKYMVME
TTNDDYWKDFMVEGKRVRTISKDAKNNTRTIIFPYVEGKALYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 14
MNKQQKEFKSFYSIRKSSLGVASVAISTLLLLMSNGEAQAAAEETGGTNTEAQPKTEAVASPTTTSEKAPETKPVAN
AVSVSNDEVEAPTSETKEAKEVKEVKAPKETKEVKPAAKATNNTYPILNQELREAIKNPAIKDKEHSAPNSRPIDFE
MKKKDGTQQFYHYASSVKPARVIFTDSKPEIELGLQSGQFWRKFEVYEGDKKLPVKLVSYDTVKDYAYIRFSVSNGT
KEVKIVSSTHFNNKEEKYDYTLMEFAQPIYNSADKFKTEEDYKAEKLLAPYKKAKTLERQVYELNKLQEKLPEKLKA
EYKKKLEDTKKALDEQVKSAVTEFQNVQPTNDKMTDLQDTKYVVYESVENNESMMDTFVKHPIKTGMLNGKKYMVME
TTNDDYWKDFMVEGQSVRTISKDAKNNTRTIIFPYIEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 15
MNKQQKEFKSFYSIRKSSLGVASVAISTLLLLMSNGEAQAAAEETGGTNTEAQPKTEAVASPTTTSEKAPETKPVAN
AVSVSNDEVEAPTSETKEAKEVKEVKAPKETKEVKPAAKATNNTYPILNQELRDAIKNPAIKDKEHSAPNSRPIDFE
MKKKDGTQQFYHYASTVKPARVIFTDTKPEIELGLQSGQFWRKFEVYEGDKKLPVKLVSYDSVKDYAYIRFSVSNGT
RAVKIVSSTHYNNKEEKYDYTLMEFAQPIYNSADKYKTEEDYKAEKLLAPYKKAKTLERQVYELNKLQDKLPEKLKA
EYKKKLDDTKKALDDQVKSAVTEFQNVQPTNEKMTDLQDTKYVVFESVENNESVMDTFVKHPIKTGMLNGKKYVVME
TTNDDYWKDFIVEGQRVRTVSKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 16
MNKQQKEFKSFYSIRKSSLGVASVAISTLLLLMSNGEAQAAAEETGGTNTEAQPKTEAVASPTTTSEKAPETKPVAN
AVSVSNDEVEAPTSETKEAKEVKEVKAPKETKEVKPAAKATNNTYPILNQELREAIKNPAIIDKDHSAPNSRPIDFE
MKKKDGTQQFYHYASSVKPARVIFTDSGPEIELGLQSGQFWRKFEVYEGDKKLPIKLVSYDTVKDYAYIRFPVSNGT
KEVKIVSSTHFNNKEEKYDYTLMEFAQPIYNSADKFKTEEDYKAEKLLAPYKKAKTLERQVYELNKIQDKLPEKLKA
EYKKKLEDTKKALDEQVKSAITEFQNVQPTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 17
MNKQQKEFKSFYSIRKSSLGVASVAISTLLLLMSNGEAQAAAEETGGTNTEAQPKTEAVASPTTTSEKAPETKPVAN
AVSVSNDEVEAPTSETKEAKEVKEVKAPKETKEVKPAAKATNNTYPILNQELREAIKNPAIIDKDHSAPNSRPIDFE
MKKKDGTQQFYHYASSVKPARVIFTDSGPEIELGLQSGQFWRKFEVYEGDKKLPIKLVSYDTVKDYAYIRFPVSNGT
KEVKIVSSTHFNNKEEKYDYTLMEFAQPIYNSADKFKTEEDYKAEKLLAPYKKAKTLERQVYELNKIQDKLPEKLKA
EYKKKLEDTKKALDEQVKSAITEFQNVQPTNEKMTDLQDTKYVVYESEENNESMMDTFVKHPIYTGMLNGKKYMVME
TTNDDYWKDFMVEGQRVRTISKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 18
MNKQQKEFKSFYSIRKSSLGVASVAISTLLLLMSNGEAQAAAEETGGTNTEAQPKTEAVASPTTTSEKAPETKPVAN
AVSVSNDEVEAPTSETKEAKEVKEVKAPKETKEVKPAAKATNNTYPILNQELREAIKNPAIIDKDHSAPNSRPIDFE
MKKKDGTQQFYHYASSVKPARVIFTDSGPEIELGLQSGQFWRKFEVYEGDKKLPIKLVSYDTVKDYAYIRFPVSNGT
KEVKIVSSTHFNNKEEKYDYTLMEFAQPIYNSADKFKDEEDYKAEKLLAPYKKAKTLERQVYELNKIQDKLPEKLKA
EYKKKLEDTKKALDEQVKSAITEFQNVQPTNEKMTDLQDTKYVVYESEENNESMMDTFVKHPIYTGMLNGKKYMVME
TTNDDYWKDFMVEGQRVRTISKDAKNNTRTIIFPYVEGKTLYDAIVKVHVKTIDYDGQYHVRIVDKEAFTKANTDKS
NKKEQQDNSAKKEATPATPSKPTPSPVEKESQKQDSQKDDNKQLPSVEKENDASSESGKDKTPATKPTKGEVESSST
TPTKVVSTTQNVAKPTTASSKTTKDVVQTSAGSSEAKDSAPLQKANIKNTNDGHTQSQNNKNTQENKAKSLPQTGEE
SNKDMTLPLMALLALSSIVAFVLPRKRKN
SEQ ID NO: 19
MNKQQKEFKSFYSIRKSSLGVASVAISTLLLLMSNGEAQAAAEETGGTNTEAQPKTEAVASPTTTSEKAPETKPVAN
AVSVSNKEVEAPTSETKEAKEVKEVKAPKETKEVKPAAKATNNTYPILNQELREGSEAIKNPAIKDKDHSAPNSRPI
DFEMKKKDGTQQFYHYASSVKPARVIFTDSKPEIELGLQSGQFWRKFEVYEGDKKLPIKLVSYDTVKDYAYIRFSVS
NGTKAVKIVSSTHFNNKEEKYDYTLMEFAQPIYNSADKFKTEEDYKAEKLLAPYKKAKTLERQVYELNKIQDKLPEK
LKAEYKKKLEDTKKALDEQVKSAITEFQNVQPTNEKMTDLQDTKYVVYESVENNESMMDTFVKHPIKTGMLNGKKYM
VMETTNDDYWKDFMVEGQRVRTISKDAKNNTRTIIFPYVEGKTLYDAIVKVHVKTIDYDGQYHVRIVDVDKEAFTKA
NTDKSNKKEQQDNSAKKEATPATPSKPTPSPVEKESQKQDSQKDDNKQLPSVEKENDASSESGKDKTPATKPTKGEV
ESSSTTPTKVVSTTQNVAKPTTASSKTTKDVVQTSAGSSEAKDSAPLQKANIKNTNDGHTQSQNNKNTQENKAKSLP
QTGEESNKDMTLPLMALLALSSIVAFVLPRKRKN

Other embodiments are within the following claims. While several embodiments have been shown and described, various modifications may be made without departing from the spirit and scope of the present invention.

1. An isolated antigen binding protein comprising a first variable region and a second variable region, wherein said binding protein binds to a target region selected from the group consisting of: mAb IG3.BD4 target region, mAb 2H2.BE11 target region, mAb 13C7.BC1 target region, and mAb 13G11.BF3 target region.

2. The binding protein of claim 1, wherein, said target region is the mAb 2H2.BE11 target region and said first variable region is a heavy chain variable (Vₕ) region comprising:
a first Vₕ complementarity determining region (CDR) comprising amino acids 26-35 of SEQ ID NO: 20 or a sequence differing from amino acids 26-35 by one amino acid;
a second Vₕ CDR comprising amino acids 50-65 of SEQ ID NO: 20 or a sequence differing from amino acids 50-65 by one amino acid; and;
a third Vₕ CDR comprising amino acids 98-107 of SEQ ID NO: 20 or a sequence differing from amino acids 98-107 by one amino acid.

3. The binding protein of claim 2, wherein said second variable region is a light chain variable (V₁) region; comprising:
a first V₁ CDR comprising amino acids 24-33 of SEQ ID NO: 21 or a sequence differing from amino acids 24-33 by one amino acid;
a second V₁ CDR comprising amino acids 49-55 of SEQ ID NO: 21 or a sequence differing from amino acids 49-55 by one amino acid; and,
a third V₁ CDR comprising amino acids 88-96 of SEQ ID NO: 21 or a sequence differing from amino acids 88-96 by one amino acid.

4. The binding protein of claim 3, wherein said binding protein is an antibody.

5. (canceled)

6. The binding protein of claim 4, wherein said Vₕ region comprises an amino acid sequence selected from the group consisting of SEQ ID NO: 20, a humanized SEQ ID NO: 20, and a de-immunized SEQ ID NO: 20; and, said V₁ region comprises an amino acid sequence selected from the group consisting of SEQ ID NO: 21, a humanized SEQ ID NO: 21, and a de-immunized SEQ ID NO: 21.

7. The binding protein of claim 4, wherein said binding protein is an antibody comprising: (a) a heavy chain comprising said Vₕ region and human hinge, CH₁, CH_{2,} and CH₃ regions from an IgG₁, IgG₂, IgG₃, or IgG₄ subtype; and, (b) a light chain comprising said V₁ region and either a human kappa C_{L} or a human lambda C_{L} region.

8. The binding protein of claim 3, wherein
said Vₕ region comprises said first Vₕ CDR consisting of amino acids 26-35 of SEQ ID NO: 20, said second. Vₕ CDR consisting of amino acids 50-65 of SEQ ID NO: 20, and said third Vₕ CDR consisting of amino acids 98-107 of SEQ ID NO: 20; and,
said V₁ region comprises said first V₁ CDR consisting of amino acids 24-33 of SEQ ID NO: 21, said second V₁ CDR consisting of amino acids 49-55 of SEQ ID NO: 21, and said third V₁ CDR consisting of amino acids 88-96 of SEQ ID NO 21.

9. The binding protein of claim 8, wherein said binding protein is an antibody.

10-12. (canceled)

13. The binding-protein of claim 9, wherein said binding protein is an antibody comprising: (a) a heavy chain consisting essentially of the amino acid sequence of SEQ ID NO: 22; and, (b) a light chain consisting essentially of the amino acid sequence. of SEQ ID NO: 23.

14-26. (canceled)

27. A pharmaceutical composition comprising the antigen binding protein of claim 1 and a pharmaceutically acceptable carrier.

28-32. (canceled)
